(19) United States
(12) Patent Application Publication
(10) Pub. No.: US 2011/0009362 A1
(43) Pub. Date: Jan. 13, 2011

(54) SOLUBILITY-ENHANCED FORMS OF APREPITANT AND PHARMACEUTICAL COMPOSITIONS THEREOF

(75) Inventors: Mahendra Ramachandra Joshi, Hyderabad (IN); Nithya Radhakrishnan, Thanjavur District (IN); Munish Kumar Dhiman, Hamirpur District (IN); Pradeep Jairao Karatgi, Hyderabad (IN); Sanjay Chhagan Wagh, Hyderabad (IN); Raviraj Sukumar Pillai, Hyderabad (IN); Harshal Prabhakar Bhagwatwar, Hyderabad (IN); Venkata Nookaraju Sreedharala, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500 016, Andhra Pradesh (IN); DR. REDDY'S LABORATORIES, INC., Bridgewater, NJ (US)

(21) Appl. No.: 12/919,331

(22) PCT Filed: Feb. 27, 2009
(86) PCT No.: PCT/US09/35394

§ 371 Date: Aug. 25, 2010

Related U.S. Application Data
(60) Provisional application No. 61/046,571, filed on Apr. 21, 2008.
(60) Provisional application No. 61/048,576, filed on Apr. 29, 2008.
(60) Provisional application No. 61/141,460, filed on Dec. 30, 2008.

(30) Foreign Application Priority Data
Feb. 27, 2008 (IN) 493/CHE/2008
Mar. 14, 2008 (IN) 643/CHE/2008
Oct. 14, 2008 (IN) 2503/CHE/2008

(51) Int. Cl. A61K 31/5377; A61K 31/724; C08B 30/18; C07D 413/06; A61P 1/08

(52) U.S. Cl. 514/58; 514/236.2; 536/103; 544/132

(57) ABSTRACT

Solubility-enhanced forms of aprepitant and processes for preparing such forms. The invention also provides solubility-enhanced forms of aprepitant that also possess stability against solid state conversions. Certain solubility-enhanced forms of aprepitant comprise a cyclodextrin or any of its derivatives. Other solubility-enhanced forms of aprepitant comprise fine particle preparations of aprepitant. The invention further provides non-nanoparticulate pharmaceutical formulations prepared using solubility-enhanced forms of aprepitant. The invention also provides taste-masked and orally disintegrating pharmaceutical formulations comprising aprepitant. Further, pharmaceutical formulations comprising solubilityenhanced forms of aprepitant and processes of preparation of such formulations, as well as methods of using them are provided.

The present invention relates to solubility-enhanced forms of aprepitant and processes for preparing such forms. The present invention also relates to solubility-enhanced forms of aprepitant comprising cyclodextrin or its derivatives. The present invention also provides taste-masked compositions for oral administration, such as orally disintegrating or dissolving dosage forms. Further, the present invention includes solubility-enhanced forms of aprepitant that also possess stability against solid state conversions. The present invention also relates to solubility-enhanced forms of aprepitant comprising fine particle preparations of aprepitant. The present invention also provides orally disintegrating pharmaceutical formulations comprising aprepitant. Further, processes of preparation of compositions comprising solubility-enhanced forms of aprepitant and pharmaceutical formulations comprising such compositions, as well as methods of using such formulations, are also provided.

Aprepitant has a chemical name 5-[[(2R,3S)-2-[(1R)-1-[3,5-bis(trifluoromethyl)phenyl]ethoxy]-3-(4-fluorophenyl)-4-morpholinyl]methyl]-1,2-dihydro-3H-1,2,4-triazol-3-one. It has structural Formula I.

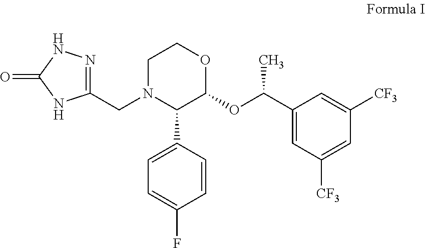

Aprepitant is a neurokinin-1 (NK-1) receptor antagonist, useful as an antiemetic agent. It is approved for the treatment of emesis associated with chemotherapy and is commercially available in the market under the brand name EMEND® as capsules containing 40 mg, 80 mg, or 125 mg of aprepitant for oral administration. Inactive ingredients contained in the capsules are sucrose, microcrystalline cellulose, hydroxypropyl cellulose, and sodium lauryl sulfate.

Aprepitant is practically insoluble in water, sparingly soluble in ethanol and isopropyl alcohol and slightly soluble in acetonitrile. Aprepitant is a molecule having poor solubility and poor permeability characteristics. Additionally, the delivery of aprepitant is also associated with high inter-patient variability when delivered as a solid dosage form, thereby requiring a nanoparticulate composition to overcome this problem. The poor solubility of aprepitant in aqueous media and poor delivery characteristics pose a tremendous challenge to the pharmaceutical formulation scientist in providing for its delivery in adequate concentrations into the systemic circulation. Some of the generally known approaches to improve drug solubility characteristics include salt formation, particle size reduction, pH adjustment, use of surfactants, inclusion complexes with cyclodextrins, use of oily formulations, use of self-emulsifying drug delivery systems, formation of co-precipitates with hydrophilic polymers, and co-milling with hydrophilic excipients, to name a few.

The rate of dissolution of poorly water-soluble drug is a rate-limiting factor in its absorption by the body. It is generally known that a reduction in the particle size of an active ingredient can result in an increase in the dissolution rate of such compounds through an increase in the surface area of the solid phase that comes in contact with the aqueous medium. There is no way to predict the extent to which the dissolution rate of an active will be enhanced through particle size reduction or what is the desirable particle size for achieving desired bioavailability characteristics. Particle size reduction beyond a certain stage may many times result in other material handling and processing issues, such as generation of static charges on new exposed surfaces and agglomeration, thereby resulting in unpredictable variation in solubility, dissolution and hence bioavailability. Use of cyclodextrins to enhance stability, aqueous solubility and bioavailability of poorly soluble drugs is known in the art. U.S. Pat. Nos. 5,070,081, 5,942,501, 6,071,964 and 6,828,334 describe methods to enhance stability and/or solubility, and bioavailability, of poorly soluble drugs with cyclodextrins.

U.S. Pat. No. 5,145,684 describes nanoparticles of pharmaceutical actives stabilized with a surface stabilizer and processes to make these particles. U.S. Patent Application Publication No. 2004/0214746 and International Application Publication No. WO 03/049718 describe nanoparticulate compositions of aprepitant and their use in the treatment of disease conditions.

In spite of the fact that the product is commercially available as a nanoparticulate composition (EMEND®) with an average particle size of less than about 1000 nm, the bioavailability of the compound when given orally is only about 60-65%. Additionally, the preparation of a nanoparticulate composition with an average particle size of less than 2000 nm is difficult and involves processing over extended periods of time using specialized equipment, making the product uneconomical to manufacture on a large scale. Also, nanoparticulate products are subject to agglomeration requiring special precautions such as addition of surface stabilizers during the processing and layering directly onto substrates to overcome these problems. Such a nanonization process could also result in physical and chemical instability of aprepitant, which is undesirable.

Conversion of a less soluble polymorphic form of an active agent to a polymorphic form having improved solubility is another approach to achieve desired release profile of active agent.

International Application Publication No. WO 2007/088483 describes preparation of amorphous aprepitant. International Application Publication No. WO 2007/112457 discloses a mixture of two crystalline forms, viz., Form I and Form II, and pharmaceutical compositions thereof. International Application Publication No. WO 2007/147160 describes compositions of amorphous aprepitant in the form of a co-precipitate that has enhanced solubility of aprepitant and composition comprising the solubility enhanced form in the form of inclusion complex.

However, none of the patents or publications in the art describes pharmaceutical compositions prepared using solubility-enhanced forms and/or solid state stable formulations or procedures to make such formulations. Hence there still remains a need for developing pharmaceutical formulations of aprepitant which have appreciable solubility, excellent solid state stability, are easy to manufacture, are cost-effective and are preferably bioequivalent with the commercial innovator product (Emend®). Also it is preferrable to prepare a pharmaceutical composition of aprepitant that is capable of releasing the drug substantially rapidly and thus making the formulation extremely useful in the treatment of emesis. The present invention alleviates the limitations of the art and provides such desirable compositions of aprepitant thus demonstrating significant advancement over the art.

### SUMMARY

The present invention relates to solubility-enhanced forms of aprepitant.

In an embodiment the invention relates to solubility-enhanced forms of aprepitant comprising cyclodextrins or its derivatives.

In an embodiment the invention includes solubility-enhanced forms of aprepitant in the form of inclusion complexes.

An aspect of the present invention provides non-nanoparticulate pharmaceutical compositions comprising solubility-enhanced forms of aprepitant which exhibit in vitro dissolution profiles that are comparable to commercially available EMEND® capsules.

An aspect of the present invention provides solubility-enhanced forms that also show excellent solid state stability of aprepitant.

In an embodiment, solubility-enhanced and solid state stable forms of aprepitant comprise aprepitant together with at least one pharmaceutically acceptable carrier.

In an embodiment, a solubility-enhanced and solid state stable form of aprepitant comprising at least one pharmaceutically acceptable carrier provides about 2-fold to about 100-fold, or about 5-fold to about 75-fold solubility enhancement in aqueous media, as compared to aprepitant alone.

In an embodiment, solubility-enhanced and solid state stable forms of aprepitant comprise aprepitant together with at least one pharmaceutically acceptable carrier, said carrier comprising at least one cyclodextrin or its analogs or derivatives.

In an embodiment, a solubility-enhanced and solid state stable form of aprepitant comprising aprepitant along with at least one cyclodextrins or its analogs or derivatives is in the form of an inclusion complex with the cyclodextrin.

In an embodiment, the solubility-enhanced forms of aprepitant in the form of inclusion complexes possess enhanced solubilities, achieved through improved complexation using a method of complexation which involves using a mixture of water and an organic solvent for complexation, and optionally using pH values other than neutral.

In one embodiment, the preparation of inclusion complex is carried out in the solution state wherein a solvent system comprises water and at least one organic solvent in volume ratios from about 1:50 to about 50:1, or from about 1:10 to about 10:1, or about 1:1, and optionally including an acidic or a basic substance to aid in the formation of a complex.

In an embodiment, a preparation of a solubility enhanced forms and solubility enhanced and solid state stable form of aprepitant comprises:

(i) preparation of a solid dispersion of aprepitant; and

(ii) preparation of an inclusion complex comprising the solid dispersion.

In a further embodiment, pharmaceutical compositions of the present invention comprise at least one disintegrating agent.

In another embodiment, pharmaceutical compositions of the present invention comprises at least two disintegrating agents, of which at least one is an ion exchange resin.

In yet another embodiment, pharmaceutical compositions of the present invention comprise at least two disintegrating agents, of which at least one is a cationic resin.

In a separate embodiment, the solubility-enhanced forms of aprepitant comprise fine particles of aprepitant which are in the form of microparticles or nanoparticles.

In one embodiment, the solubility-enhanced forms of aprepitant are in the form of nanoparticulate co-precipitates of aprepitant.

In another embodiment, the fine particles comprise aprepitant in the form of co-precipitates along with a pharmaceutically acceptable carrier, wherein the aprepitant co-precipitate has an average particle size of less than about 100 μm.

In another aspect, an aprepitant co-precipitate is present in the form of nanoparticulates with an average particle size less than about 2000 nm.

In embodiments, the solubility-enhanced complexed aprepitant shows about 5-fold to about 200-fold, or from about 20-fold to about 150-fold solubility enhancement in aqueous media, when compared with uncomplexed aprepitant.

The present invention also relates to processes for preparing solubility-enhanced forms of aprepitant in the form of fine particles.

Aspects of the present invention also provide pharmaceutical compositions comprising solubility-enhanced forms or solubility enhanced and solid state stable forms of aprepitant.

In yet another embodiment, pharmaceutical formulations of the present invention are appreciably stable and easy to manufacture using conventional processing steps, as compared to the currently marketed nanoparticulate formulations of aprepitant (EMEND®) that are prepared using difficult manufacturing steps and the use of specialized machinery, but still provides in vitro and in vivo release profiles of aprepitant that are comparable to those of EMEND® capsules.

In one embodiment, pharmaceutical formulations of the present invention comprising solubility-enhanced forms, and solubility-enhanced and solid state stable forms of aprepitant provide in vitro dissolution of aprepitant such that more than about 90% of the drug is dissolved within 60 minutes after immersion into 900 ml of a 2.2% w/v sodium lauryl sulphate aqueous solution, wherein the dissolution is conducted using USP Type II (paddle type) apparatus with 75 RPM stirring.

In an aspect, bioequivalent compositions of aprepitant comprise solubility-enhanced forms of aprepitant that are in the form of an inclusion complex or microparticulate or nanoparticulate co-precipitates.

In another aspect, bioequivalent compositions of aprepitant comprises solubility-enhanced and solid state stable forms of aprepitant that contain at least one cyclodextrin or its analog or derivative, and are in the form of an inclusion complex.

In an embodiment of the present invention, solubility-enhanced forms or solubility-enhanced and solid state stable forms of aprepitant are used for preparation of orally disintegrating or orally dissolving compositions of aprepitant.

In a further aspect, the invention relates to preparation of orally disintegrating or orally dissolving tablet compositions that contain aprepitant along with at least one cyclodextrin or its analog or derivative and are in the form of inclusion complexes.

In another embodiment the present invention provides processes of preparation of orally disintegrating or dissolving tablet compositions of aprepitant.

Further the invention provides in-vitro dissolution profiles of orally disintegrating tablet compositions of aprepitant.

In an aspect, the present invention provides taste-masked compositions of aprepitant for oral administration, as orally disintegrating or dissolving dosage forms.

The invention further provides conversion of these solubility-enhanced forms, and solubility-enhanced and solid state stable forms of aprepitant into pharmaceutical compositions that help in the effective delivery of aprepitant or its isomers.

The invention also provides methods of using the pharmaceutical compositions comprising solubility-enhanced forms of aprepitant by administration to a subject in need thereof, particularly in the treatment of chemotherapy induced nausea and vomiting.

An aspect of the invention includes a solid state non-nanoparticulate solubility-enhanced form of aprepitant comprising aprepitant as a co-precipitate, premix, or a solid dispersion with at least one pharmaceutically acceptable carrier, wherein the said co-precipitate, premix, or a solid dispersion of aprepitant is prepared in the form of an inclusion complex with at least one cyclodextrin or cyclodextrin derivative.

An aspect of the invention includes a process for preparing an inclusion complex of aprepitant with a cyclodextrin, or a derivative of a cyclodextrin, comprising combining aprepitant or a co-precipitate, premix, or a solid dispersion of aprepitant with a cyclodextrin or cyclodextrin derivative in a solution having a pH within the ranges of about 1 to about 5, or about 8 to about 12.

An aspect of the invention includes a pharmaceutical formulation comprising a solid state stable non-nanoparticulate solubility-enhanced form of aprepitant, wherein more than about 30% of the aprepitant is dissolved within 60 minutes after immersion in 900 ml of fed state simulated intestinal fluid pH 5.0 dissolution medium, when tested in USP apparatus II at 75 rpm stirring.

An aspect of the invention includes a pharmaceutical formulation comprising a solid state stable non-nanoparticulate solubility-enhanced form of aprepitant, wherein about 15% to about 60% of the aprepitant is dissolved within about 15 minutes, about 25% to about 70% of the aprepitant is dissolved within about 30 minutes, about 35% to about 75% of the aprepitant is dissolved within about 45 minutes, and about 40% to about 90% of the aprepitant is dissolved within about 60 minutes, after immersion into 900 ml of fed state simulated intestinal fluid pH 5.0 dissolution medium, when tested in USP apparatus II at 75 rpm stirring.

An aspect of the invention provides an orally disintegrating pharmaceutical formulation, containing a solubility-enhanced form of aprepitant, that disintegrates in less than about 10 minutes upon immersion in water.

### BRIEF DESCRIPTION OF THE DRAWINGS

FIG. 1 shows comparative X-ray powder diffraction (“XRD”) patterns of a solid state stable formulation (B) prepared using a spray drying process (Example 4A), a placebo formulation (C) that is devoid of aprepitant (Example 4B), both as initially prepared, and crystalline Form I of aprepitant (A).

FIG. 2 shows comparative XRD patterns of a solid state stable formulation (B) prepared using a spray drying process (Example 4A), a placebo formulation (C) that is devoid of aprepitant (Example 4B), both after 1 month of storage at 25° C. and 60% relative humidity (“RH”), and crystalline Form I of aprepitant (A).

FIG. 3 shows comparative XRD patterns of a solid state stable formulation (B) prepared using a spray drying process (Example 4A), a placebo formulation (C) that is devoid of aprepitant (Example 4B), both after 1 month of storage at 40° C. and 75% RH, and crystalline Form I of aprepitant (A).

FIG. 4 shows comparative XRD patterns of a solid state stable formulation (B) prepared using a fluidized bed coating process (Example 5B), a placebo formulation (C) that is devoid of aprepitant (Example 5E), both as initially prepared, and crystalline Form I of aprepitant (A).

FIG. 5 shows comparative XRD patterns of a solid state stable formulation (B) prepared using a fluidized bed coating process (Example 5B), a placebo composition (C) that is devoid of aprepitant (Example 5E), both after 1 month of storage at 25° C. and 60% RH, and crystalline Form I of aprepitant (A).

FIG. 6 shows comparative XRD patterns of a solid state stable formulation (B) prepared using a fluidized bed coating process (Example 5B), a placebo composition (C) that is devoid of aprepitant (Example 5E), both after 1 month of storage at 40° C. and 75% RH, and crystalline Form I of aprepitant (A).

### DETAILED DESCRIPTION

The present invention relates to solubility-enhanced forms of aprepitant and processes for preparation thereof. The present invention also relates to solubility-enhanced forms of aprepitant that possess excellent solid state stability. Embodiments of solubility-enhanced forms, and solubility-enhanced and solid state stable forms, of aprepitant comprise cyclodextrin or its derivatives.

In an aspect, the present invention provides non-nanoparticulate pharmaceutical compositions comprising solubility-enhanced forms of aprepitant that exhibit in vitro dissolution profiles comparable to commercially available EMEND® capsules.

In an aspect, the solubility-enhanced forms of aprepitant are in the form of inclusion complexes which possess enhanced solubilities, achieved through improved complexation using a method of complexation which involves using a mixture of water and an organic solvent for complexation and optionally pH values other than neutral.

In one embodiment, the solubility-enhanced forms of aprepitant are in the form of nanoparticulate co-precipitates of aprepitant.

In an aspect, the present invention provides pharmaceutical compositions of aprepitant that are bioequivalent to EMEND® capsules, and which comprise solubility-enhanced forms of aprepitant that are in the form of an inclusion complex or microparticulate or nanoparticulate co-precipitates.

In another aspect, solubility-enhanced forms of aprepitant comprise fine particles of aprepitant including microparticles of crystalline aprepitant, and micro- and nanoparticles of amorphous aprepitant alone or in the form of a co-precipitate. In embodiments, average particle sizes of aprepitant fine particles are less than about 500 μm and comprise aprepitant alone or in combination with one or more pharmaceutically acceptable excipients. Further the present invention relates to pharmaceutical compositions comprising solubility-enhanced forms as well as solubility-enhanced and solid state stable forms of aprepitant and process of preparation thereof, and methods of using such compositions.

Unless mentioned otherwise, all embodiments of the invention can be used for the delivery of aprepitant or any of its pharmaceutically acceptable salts, solvates, enantiomers or mixtures thereof, without limitation.

In one aspect, the term “aprepitant” includes an amorphous form, alone or in combination with any other polymorphic form, crystalline Forms I or II, or includes a combination of crystalline Forms I and II, co-precipitates, premixes, solid dispersions, and the like.

Further, the invention relates to solubility-enhanced forms of aprepitant with improved solubility characteristics that help in the effective delivery of aprepitant.

The term “co-precipitate” as used in this invention refers to compositions comprising aprepitant in intimate mixture with at least one pharmaceutically acceptable carrier. The intimate mixtures differ from simple physical mixtures of powders, in that particles of the individual components cannot be identified using techniques such as optical microscopy.

An aspect of the present invention includes methods of preparation of co-precipitate compositions of aprepitant with pharmaceutically acceptable carriers, a specific embodiment comprising the steps of:

a) Providing a solution of aprepitant and a pharmaceutically acceptable carrier.

b) Removing the solvent.

c) Optionally, drying the solid obtained to obtain the co-precipitate.

The term “pharmaceutical composition/formulation” as used herein refers to compositions comprising an aprepitant solubility-enhanced form as described herein together with one or more pharmaceutically acceptable excipients as required to convert the solubility-enhanced form of aprepitant into dosage forms for the effective delivery of aprepitant.

The term “solubility property” as used herein refers to either an improvement in the solubility of aprepitant, or a modification in the rate of dissolution or a modified absorption of aprepitant.

The “solubility-enhanced form” or “enhanced solubility form” of aprepitant refer to aprepitant with improved solubility properties that have a higher solubility and/or dissolution rate, as compared to aprepitant in its crystalline form. Unless specified otherwise, the solubility-enhanced forms include solubility-enhanced forms as well as solubility-enhanced and solid state stable forms of aprepitant.

The term “solid state stable form” or “stable form” or “appreciably stable form” as used herein refers to a solid state of aprepitant that is less likely to change its physical or chemical form upon storage during the storage life; or in case of conversion, such conversion does not lead to alteration of the characteristics (physical, chemical, biological, pharmaceutical or the like) of aprepitant. The solid state stable forms may include crystalline form I, crystalline form II or any other crystalline form, and an amorphous form, also including premixes, co-precipitates, solvates and the like, or a mixture of amorphous and one or more crystalline forms.

Improved solubility properties of aprepitant according to the present invention can also be obtained by use of emulsifiers, solubilizers, co-precipitates or solid dispersions, premixes, inclusion and other complexes, use of amorphous or alternate crystalline forms, and the like, including combinations thereof, in pharmaceutical compositions.

Further, the invention provides solubility-enhanced forms comprising aprepitant in the form of complexes.

In an aspect of the invention, solubility-enhanced forms of aprepitant are provided wherein aprepitant and a cyclodextrin or its derivative forms inclusion complexes.

In a further aspect of the invention, crystalline form of aprepitant is used for preparing inclusion complexes with cyclodextrins.

In another aspect of the invention, amorphous co-precipitates of aprepitant are used for preparing inclusion complexes with cyclodextrins.

According to an aspect of the present invention, solubility-enhanced and solid state stable forms of aprepitant comprise aprepitant together with at least one pharmaceutically acceptable carrier, wherein the carrier comprises at least one cyclodextrin or its analogs or derivatives.

In an aspect, a solubility-enhanced and solid state stable form of aprepitant is in the form of an inclusion complex.

A solubility-enhanced form as well as solubility-enhanced and solid state stable form of aprepitant of embodiments of the present invention provides about 2-fold to about 100-fold, or from about 5-fold to about 75-fold, solubility enhancement as compared to crystalline aprepitant alone.

In one embodiment, the preparation of an inclusion complex according to the present invention is carried out in a solution state wherein the solvent system comprises water and at least one organic solvent in a volume ratio from about 1:10 to about 10:1, or about 1:1, and optionally adding a acidic or a basic substance to promote the formation of a complex.

According to an embodiment of the present invention, preparation of a solubility-enhanced and solid state stable form of aprepitant comprises steps of:

(i) preparation of a solid dispersion of aprepitant; and

(ii) preparation of an inclusion complex comprising the solid dispersion.

In yet another aspect, pharmaceutical compositions of the present invention comprise solid dispersions of aprepitant, wherein a solid dispersion of aprepitant is provided as a co-precipitate, premix, or co-crystals, or adsorbed onto at least one pharmaceutically acceptable carrier. Carriers according to the present invention include but are not limited to polyvinylpyrrolidones (povidone or PVP), hydroxypropyl methylcelluloses (hypromellose or HPMC), sugars such as mannitol, sorbitol, etc., and the like. Optionally, a solid dispersion comprises one or more antioxidants.

As used herein, “cyclodextrin” refers to any of the natural cyclodextrins, α-cyclodextrin, β-cyclodextrin, and γ-cyclodextrin, and their respective derivatives or analogs. Cyclodextrins (sometimes called cycloamyloses) make up a family of cyclic oligosaccharides, composed of 5 or more α-D-glucopyranoside units linked 1→4, as in amylose (a fragment of starch). The formation of the inclusion compounds greatly modifies the physical and chemical properties of the guest molecules (such as aprepitant in the present invention), mostly in terms of water/aqueous solubility. An inclusion complex of aprepitant with cyclodextrins also aids in penetration of the drug into body tissues.

Any cyclodextrin, which enhances the aqueous solubility and/or provides for effective delivery of aprepitant, may be used in the present invention. The cyclodextrins of the present invention can include the natural occurring cyclodextrins and their derivatives. The natural cyclodextrins include α-cyclodextrin, β-cyclodextrin and γ-cyclodextrin. Derivatives are typically prepared by modifying the hydroxyl groups located on the exterior or hydrophilic side of the cyclodextrin. The modifications can be made to increase the aqueous solubility and the stability of the complexes and can modify the physical characteristics of the complexes, including the formation and dissociation of the complex. The types and degrees of modification, as well as their preparation, are well-known in the art.

Any of the natural cyclodextrins can be derivatized, such as derivatives of β-cyclodextrin. Cyclodextrin derivatives include alkylated cyclodextrins, comprising methyl-, dimethyl-, trimethyl- and ethyl-β-cyclodextrins; hydroxyalkylated cyclodextrins, including hydroxyethyl-, hydroxypropyl-, and dihydroxypropyl-β-cyclodextrin; ethyl carboxymethyl cyclodextrins; sulfate, sulfonate and sulfoalkyl cyclodextrins, such as β-cyclodextrin sulfate, β-cyclodextrin sulfonate, and β-cyclodextrin sulfobutyl ether; as well as polymeric cyclodextrins. Other cyclodextrin derivatives can be made by substitution of the hydroxy groups with saccharides, such as glucosyl- and maltosyl-β-cyclodextrin.

Other cyclodextrins include the naturally occurring cyclodextrins, methyl-β-cyclodextrin, dimethyl-β-cyclodextrin, trimethyl-β-cyclodextrin, 2-hydroxymethyl-β-cyclodextrin, hydroxyethyl-β-cyclodextrin, 2-hydroxypropyl-β-cyclodextrin, 3-hydroxypropyl-β-cyclodextrin, β-cyclodextrin sulfate, β-cyclodextrin sulfonate, or β-cyclodextrin sulfobutyl ether. Any of the above cyclodextrins or their derivatives or polymers prepared from them can be used for preparation of the compositions of the invention, either alone or in the form of mixtures of one or more cyclodextrins.

Commercially available cyclodextrins may be used such as those available from any of the commercial suppliers such as for example Cargill Inc, Wayzata, Minn. USA, Roquette Freres, Lestrem, France, Aldrich Chemical Company, Milwaukee, Wis. USA and Wacker Chemicals, New Canaan, Conn. USA, or the cyclodextrins may be synthesized by any of the processes known in the art for the synthesis of cyclodextrins and their derivatives.

In another aspect, the complexation is complete or partial, or aprepitant and the cyclodextrin exist together in intimate contact as a powder, and result in a clear solution comprising the aprepitant after contact with a bio-relevent medium (in situ complex). Thus, according to this embodiment the aprepitant or co-precipitate are processed together with a cyclodextrin to form an inclusion complex.

In one of the embodiments the invention includes use of hydroxypropyl-β-cyclodextrin (“HPβCD”) for complexation with aprepitant.

In a further aspect of the invention, crystalline form such as Form I of aprepitant is used for preparing inclusion complexes of aprepitant with cyclodextrins. In another aspect of the invention, amorphous co-precipitates or substantially amorphous co-precipitates of aprepitant are used for preparing inclusion complexes with cyclodextrins.

Weight ratios of aprepitant to cyclodextrin may be from about 1:0.01 to about 1:200, or from about 1:0.1 to about 1:100, or from about 1:0.25 to about 1:50, in the compositions of the invention.

When the amount of the aprepitant present is more than an amount that can be incorporated into the inclusion complex using the amount of cyclodextrin selected, the remaining drug substance will be present in the form of a crystalline or amorphous drug substance as part of the mixture. Such solubilizing compositions are also within the scope of the invention without limitation. The amount of such free or uncomplexed drug present within the powder composition will be determined by the amount and type of the cyclodextrin, the complexation capacity of the cyclodextrin selected, the process utilized to prepare the powder composition, and other parameters known to a person skilled in the art.

Cyclodextrins with lipophilic inner cavities and hydrophilic outer surfaces are capable of interacting with a large variety of guest molecules to form non-covalent inclusion complexes. The stability of the complex formed depends on how well the guest molecule fits into the cyclodextrin cavity. Without being bound by theory, it is felt that the processing of the lipophilic active along with the cyclodextrin provides a composition wherein the active is in intimate contact with the cyclodextrin though not in the form of an inclusion complex. Thus, upon coming in contact with bio-relevant media, the active is forced into solution along with the cyclodextrin.

It is frequently desirable that the aprepitant be present as an inclusion complex with very little or no free or uncomplexed drug present in the solubilizing compositions of the invention. Thus, according to this aspect of the invention, aprepitant in the solubility-enhanced form is at least about 60%, or about 75%, or about 80%, or about 85%, or about 90%, or about 95%, or about 100%, complexed in the solubilizing compositions of the invention.

The uncomplexed drug, when present in a solubility-enhanced form of the invention, can be in a crystalline form, or in amorphous form, or mixtures thereof. The crystalline form can be the same as the one used in the preparation of the solubility-enhanced form, or a different crystalline form or a mixture of forms could be present.

In another aspect of the invention, the aprepitant present in the solubility-enhanced form of the invention is at least about 50%, or at least about 75%, or at least about 80%, or at least about 85%, or at least about 90%, or at least about 95%, or about 100%, amorphous. Percentages of amorphous content can be determined by X-ray powder diffraction and the amorphous form will be characterized by an absence of peaks which identify aprepitant crystalline forms. Without being bound by any particular theory, the degree of amorphous content can be an indication of the completeness of complexation of the crystalline form, though uncomplexed amorphous aprepitant could also be present in the powder compositions.

Various methods are known in the art to prepare drug-cyclodextrin complexes, including the solution method, co-precipitation method, the slurry method, the kneading method, and the grinding method. See T. Loftsson, “Pharmaceutical Applications of β-Cyclodextrin,” Pharmaceutical Technology, Vol. 23(12), pages 40-50, 1999.

In a solution method, the drug, either as a solid or in a solution, is added to a solution containing an excess amount of cyclodextrin. It is also possible to add an excess of the drug to an aqueous cyclodextrin solution. The mixture is agitated, and may optionally be heated, until equilibrium is reached, which may take several hours or several days. The equilibrated solution is then filtered or centrifuged to give a clear solution of the drug-cyclodextrin complex. The clear solution can be directly administered to a subject, or a solid complex can be obtained by removal of the water by evaporation (such as spray-drying), sublimation (such as lyophilization) or other drying means well known in the art.

A solid complex may also be obtained by a precipitation method. Often, cyclodextrin complexes precipitate upon cooling of their solution. Otherwise, a solvent in which the complex has minimal solubility, typically an organic solvent, can be used to precipitate a solid complex. A precipitate containing the complex can then be filtered or centrifuged to obtain a solid drug-cyclodextrin complex. A generally less effective method of preparing a solid complex mixture is to grind a dry mixture of the drug and cyclodextrin in a sealed container, which is then gently heated to a temperature between about 60° C. and 140° C.

Further, slurry or kneading methods can also be employed. The drug and cyclodextrin can be suspended in water to form a slurry, which is similarly stirred and/or heated to equilibration. The complex can be collected by filtration or by evaporation of the water. The kneading method is similar to the slurry method, whereby the drug and cyclodextrin are mixed with a minimal amount of water to form a paste. The complex can be isolated by methods similar to those discussed hereinabove.

The above methods generally utilize an excess amount of cyclodextrin to maximize equilibration of a cyclodextrin-drug complex. The amount of cyclodextrin in the desired formulation is directly related to the desired drug concentration and the molar ratio of cyclodextrin to drug in the complex.

Any method can be used for the preparation of the inclusion complexes of the invention including but not limited to the methods described above. According to an aspect of the invention, processes for the preparation of the inclusion complexes of the invention are provided comprising combining a cyclodextrin and aprepitant in the desired ratio under suitable conditions, optionally along with other pharmaceutically acceptable excipients that aid or enhance the complexation or act as bulking agents.

In embodiments of the invention, water or aqueous solutions, or mixtures of water with water-miscible organic solvents, are used as solvent system for the preparation of the inclusion complexes. Any solvent system is acceptable for the preparation of the inclusion complexes of the invention as long as the aprepitant is soluble or dispersible in the solvent system, the cyclodextrin is soluble in the solvent system and the solvent system is not chemically detrimental to the aprepitant or the complex formed.

The solvent systems used in the preparation of the inclusion complexes include but are not limited to water, methanol, ethanol, acidified ethanol, acetone, diacetone, polyols, polyethers, oils, esters, alkyl ketones, acetonitrile, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, tetrahydrofuran, N,N-dimethylformamide, and mixtures of any two or more thereof.

In an embodiment, the preparation of inclusion complexes is carried out in a solution state, wherein the solvent system comprises water or an organic solvent, or a combination of water and at least one organic solvent. In an aspect, the solvent system comprises combinations of water and at least one organic solvent, in volume ratios from about 1:50 to about 50:1, or about 1:10 to about 10:1, or about 1:1.

In an embodiment, the preparation of an inclusion complex is carried out in a solution state wherein a solvent system comprises water and at least one organic solvent in volume ratios from about 1:10 to about 10:1, or about 1:1, and optionally an acidic or a basic substance to promote the formation of a complex.

In an embodiment, the complexation can be carried out in the pH range about 1 to about 4, or about 1 to about 5, including use of an acidic solution such as 0.1N HCl, or in a pH environment above about 8, or about 8 to about 12, such as in a solution of a basic substance such as sodium carbonate.

The ratios of the solvent medium to the aprepitant may be determined by the final concentration of the aprepitant, which is to be achieved in solution in the form of a complex, and the cyclodextrin that is to be used. As a routine practice, solutions of the cyclodextrin in the solvent medium, in water for example, are prepared in desired concentrations. To these solutions are added desired amounts of aprepitant and the suspensions are allowed to equilibrate, aided by shaking. The suspensions are subsequently filtered and analyzed for their aprepitant content. The temperature of the solvent medium is usually kept at about ambient temperature, although higher or lower temperatures may be used as required. Any temperature is acceptable as long as it is not detrimental to the chemical stability of the active and the cyclodextrin, and to the stability of the inclusion complex formed.

In an aspect, the invention provides processes for preparing solubility-enhanced forms and solubility-enhanced and solid state stable forms of aprepitant, wherein an embodiment of a process comprises:

a) Preparing an aqueous solution of cyclodextrin.

b) Adjusting the pH of the cyclodextrin solution using a desired pH modulator.

c) Adding aprepitant to the cyclodextrin solution with constant stirring or sonication until solubility is achieved.

d) Optionally filtering the solution.

e) Recovering a solubility-enhanced or solubility-enhanced and solid state stable form of aprepitant from the solution.

In a specific embodiment the invention includes processes to prepare solubility-enhanced and solubility-enhanced and solid state stable forms of the invention comprising:

a) Providing a solution or dispersion comprising aprepitant and a cyclodextrin in a suitable solvent medium.

b) Adjusting the pH of the solution of step (a) as desired, using a pH modulator.

c) Recovering a solubility-enhanced form of aprepitant from the solution.

An aspect of the invention includes processes to prepare solubility-enhanced forms in the form of inclusion complexes of aprepitant, wherein an embodiment of a process comprises:

a) Providing a dispersion of aprepitant in a suitable solvent medium.

b) Optionally adding a pharmaceutically acceptable bulking agent.

c) Adding complexation enhancers to the dispersion of step (a) or step (b) and optionally adjusting the pH as desired.

d) Dissolving a cyclodextrin in the dispersion of step (c).

e) Mixing the dispersion of step (d) to form a clear solution.

f) Adjusting the pH of the clear solution of step (e) as desired using a pH modulator.

g) Optionally filtering the solution.

h) Optionally evaporating the solvent to obtain a dry product.

It is desirable, though not essential, that the aprepitant has particle sizes as small as possible before being added to the solvent medium. Smaller particle sizes enhance the speed of dissolution of a solid in a given solvent medium. Also, smaller particle sizes enhance the suspendability in the medium when the method of preparation of the inclusion complex involves the preparation of a dispersion of the active in the solvent medium. In addition, smaller particle sizes also reduce the time of complexation.

The particle sizes may be reduced to the desired level by any method of size reduction known in the art such as for example pulverization, jet milling (using a compressed gas), ball milling, and the like without limitation. Alternatively, larger particles can be added to the medium and the slurry can be subjected to homogenization using for example a high speed homogenizer, a high pressure homogenizer, colloid milling, Emulsiflex, microfluidizer, bead mill, and the like without limitation. Other methods of size reduction are well within the scope of this invention without limitation.

The particle size distribution of a material is generally described in terms of D₁₀, D₅₀, D₉₀, and D[4,3], used routinely to describe the particle sizes or size distributions. It is expressed as volume or weight or surface percentages. Dₓ as used herein is defined as the size of particles where x percent of the particles have sizes less than the value given. D[4,3] is the volume mean diameter of the particles. D₉₀ for example means that 90% of the particles are below the specified particle size. Particle sizes or particle size distributions of the pharmaceutical compositions of aprepitant of the present invention are determined using any techniques that are known to the person skilled in the art including but not limited to sieve analysis, size analysis by laser light scattering such as using a Malvern particle size analyzer (Malvern Instruments Ltd., Malvern, Worcestershire, United Kingdom) and the like. Pharmaceutical compositions of aprepitant of the present invention are generally fine, uniform and free of agglomerates.

As used herein, the term “mean particle size” refers to the distributions of aprepitant particles, including aprepitant co-precipitate particles, wherein about 50 percent of all particles measured have a particle size less than the defined mean particle size value and about 50 percent of all measurable particles measured have a particle size greater than the defined mean particle size value; this can be identified by the term “D₅₀.” Similarly, a particle size distribution where 90 percent of the particles have sizes less than a specified size is referred to as “D₉₀” and a distribution where 10 percent of particles have sizes less than a specified size is referred to as “D₁₀.”

According to embodiments of the invention, “non-nanoparticulate” aprepitant particles, including aprepitant co-precipitate particles, have particle sizes greater than 3 μm, or particle sizes greater than 3 μm and less than about 500 μm. In embodiments, non-nanoparticulate particles are greater than about 5 μm or greater than about 10 μm, and less than about 500 μm.

According to embodiments, nanoparticulate particles of aprepitant, including aprepitant co-precipitate particles, according to the present invention have D₅₀ less than about 500 nm and D₉₀ less than about 2000 nm.

According to embodiments, microparticulate particles of aprepitant, including aprepitant co-precipitate particles, according to the present invention are greater than 3 μm, and have D₅₀ less than about 100 μm and D₉₀ less than about 500 μm.

The processes for preparing the solubility-enhanced forms can further involve the addition of a pharmaceutically acceptable bulking agent, and addition of complexation enhancers as desired.

The processes of preparing the solid state stable forms can further involve the addition of one or more pharmaceutically acceptable excipients including, for example, wetting agents, surfactants, co-surfactants, pH modulators, diluents or bulking agents, binders, complexation enhancers, and the like. Some of the excipients included may be capable of having more than one role in the preparation of the solubilizing compositions. Such pharmaceutically acceptable excipients may be added to the solvent medium before the addition of aprepitant or can also be added to the dispersion prepared. Complexation enhancers may be in the form of surfactants, alkalizing agents, acidifying agents, solubilizers, and mixtures thereof.

Bulking agents can reduce drug loss during processes such as spray drying. Further, the presence of a bulking agent is useful in modifying the physicochemical properties of the pharmaceutical compositions such as bulk density, which affects the amount of active that can be incorporated into the pharmaceutical delivery vehicle such as for example a capsule. Additionally, the inclusion of a suitable pharmaceutically acceptable bulking agent allows the preparation of a product, which is ready to fill into capsules or compress into tablets, with appropriate flow properties and compressibility. In the case of a lyophilized product, for example, the bulking agent allows the final solution of the inclusion complex to be lyophilized to provide a product with aesthetic appeal. Suitable pharmaceutically acceptable bulking agents include but are not limited to mannitol, sodium chloride, sucrose, glucose, lactose, dextrose, dextrins, and the like, and mixtures thereof.

In an embodiment, the invention includes the use of complexation enhancers that are either acidifying or alkalizing agents. Useful acidifying agents include but are not limited to fumaric acid, tartaric acid, citric acid, malic acid, succinic acid, ascorbic acid, and mixtures of any of these acids, as well as pharmacologically acceptable acid substances such as the acid salts sodium or potassium hydrogen sulphate, monosodium or monopotassium salts of polybasic acids (e.g., tartaric acid or citric acid), and mixtures thereof.

Alkalizing agents that can act as complexation enhancers include but are not limited to organic amines such as meglumine, tromethamine, triethanolamine, diethanolamine, etc., inorganic alkaline substances such as for example sodium hydroxide, sodium carbonate, sodium bicarbonate and the like, and amino acids such as alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine and the like. The use of mixtures of two or more of the above mentioned alkalizing agents, either from the same class or from different classes of alkalizing agents, is also within the scope of the invention.

In one aspect, the pH of the dispersion is optionally adjusted to be in a desired range. An alkaline or acidic pH is generally desirable due to the high aqueous solubility of aprepitant in alkaline or acidic conditions as compared with the neutral pH. Any pH is acceptable as long as it is not detrimental to the chemical stability of aprepitant. Any of the alkalizing or acidifying agents mentioned above can be used for adjusting the pH in the desired range.

Embodiments of processes of making inclusion complexes according to the present invention involve making a dispersion of aprepitant and cyclodextrin optionally along with one or more pharmaceutically acceptable excipients in a solvent system, and mixing the dispersion to form a clear solution. Any means of mixing dispersions is acceptable as long as it provides a clear solution of the aprepitant in the aqueous medium. Such mixing means could include for example overhead stirrers, homogenizers, static mixers, sonicators and the like. The duration of mixing will be decided based on parameters such as concentration to be achieved, the temperature of the dispersion, the type of cyclodextrin, the mixing means, the particle size of the aprepitant or its individual isomers in the dispersion and such other parameters known to a person skilled in the art of preparing inclusion complexes. The temperature of the dispersion may be increased to enhance the rate of formation of the inclusion complex. A temperature in the range of about 20° C. to about 70° C., or about 20° C. to about 40° C., is generally acceptable, though lower or higher temperatures are well within the scope of the invention. When uncomplexed drug is not desired, ensure that a clear solution is achieved before the mixing is discontinued, as this is an indication of completeness of formation of the inclusion complex. Finally, the clear complex solution can be filtered and the solvent evaporated to obtain a dry product of a solubility-enhanced form of aprepitant. The clear complex solution obtained as described above may be filtered to remove extraneous material or undissolved drug substance to prevent these from getting into the final product. Any filter medium may be chosen such as for example different grades of membrane filters, sintered glass filters, and the like.

The filtered solution may optionally be subjected to evaporation of the solvent medium to recover a dry product. Any method of solvent evaporation or drying is acceptable as long as it is not detrimental to the chemical stability of the drug as well as the solubilizing composition. Such methods could include for example tray drying, vacuum drying, spray drying, spray coating, lyophilization, microwave drying and the like without limitation. Two or more methods could be used sequentially to ensure completeness of removal of the solvent medium or to achieve desirable bulk properties of the dried solubilizing compositions. Thus, according to one particular embodiment, the inclusion complex solution as prepared above is spray dried and the resulting powder is optionally further subjected to vacuum drying to get a desired moisture content.

According to an embodiment of the invention, the inclusion complex solution as prepared above is further subjected to lyophillization to obtain a dry product which constitutes one of the powder compositions of the invention. Lyophillization is a drying technique of particular interest in the preparation of dry powder compositions of the invention due to its rapid drying cycles, high throughputs, scalability and short exposure times to high temperatures, achievement of desired bulk properties, and other reasons.

In an aspect, the invention provides processes for preparing stable and solubility-enhanced forms of aprepitant, wherein an embodiment of a process comprises:

a) preparing an aqueous solution of a cyclodextrin;

b) preparing a solution of aprepitant using an organic solvent;

c) mixing solutions of a) and b) with continuous stirring or sonication;

d) dissolving a carrier in the solution of c);

e) optionally filtering the solution; and

f) drying the solution from d) or e) using a spray dryer to obtain the desired product.

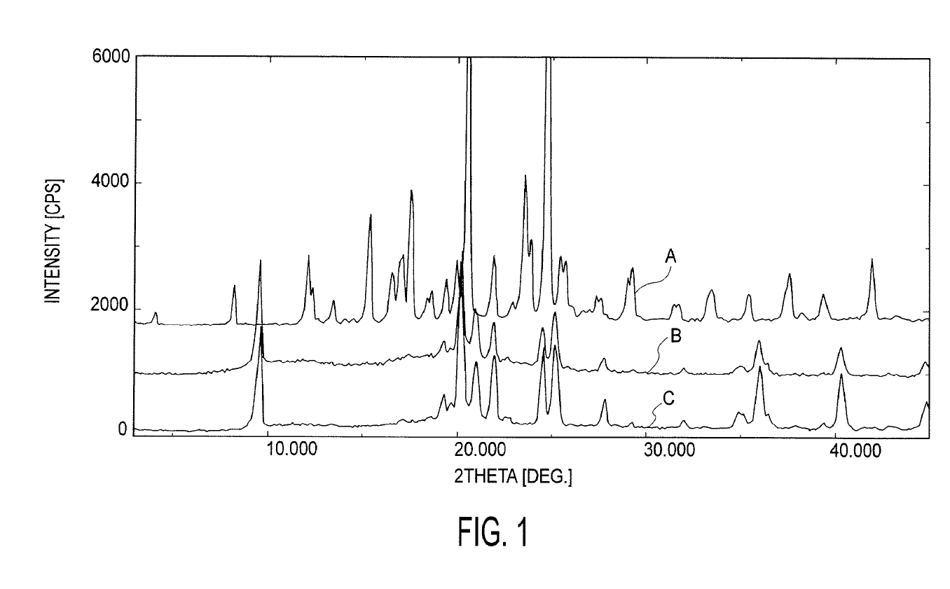

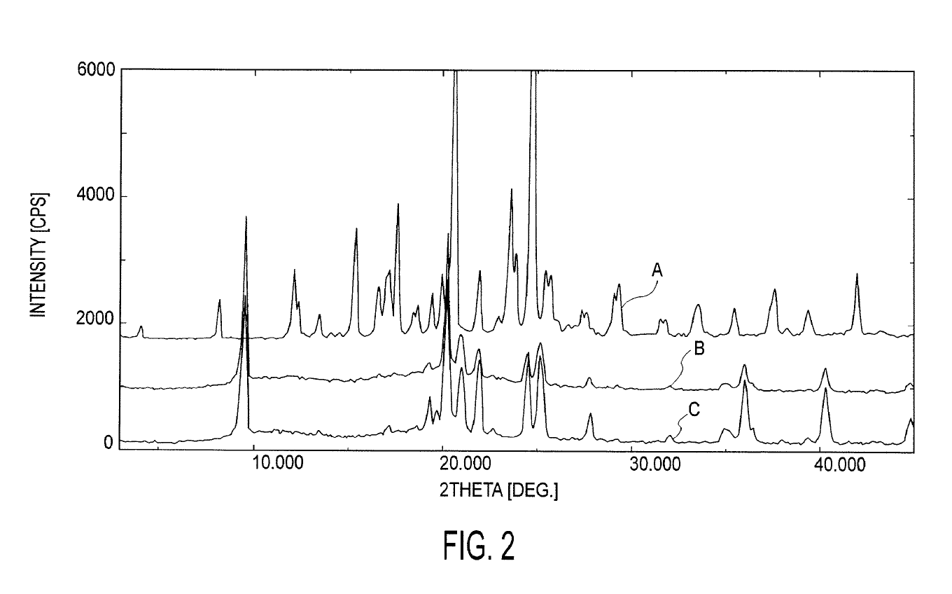

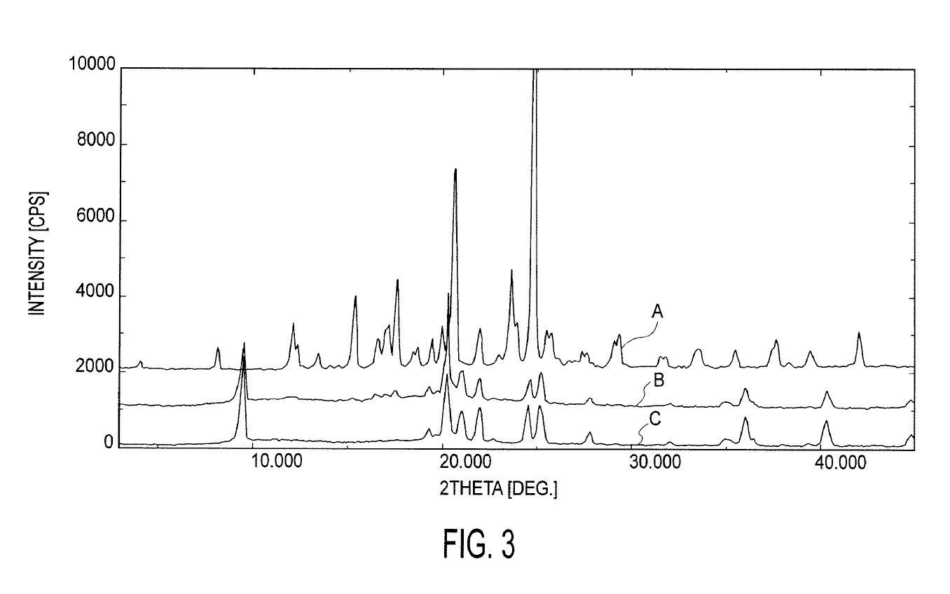

In an aspect, spray dried stable and solubility-enhanced forms of aprepitant are subjected to storage stability testing at 25° C. and 60% RH, and 40° C. and 75% RH, for a commercially relevant time. The samples were examined by X-ray diffraction and are compared with crystalline Form I and a placebo formulation. No peaks pertaining to crystalline Form I are detected, indicating absence of crystallinity in the pharmaceutical composition comprising the amorphous aprepitant co-precipitate (see FIGS. 1-3). Such solid state stability ensures the maintenance of enhanced solubility throughout the commercial shelf life of the formulation.

In an aspect, the invention provides processes to prepare stable and solubility-enhanced forms of aprepitant in the form of inclusion complexes, wherein an embodiment of a process comprises:

a) preparing an aqueous solution of cyclodextrin;

b) preparing a solution of aprepitant using an organic solvent;

c) mixing solutions of a) and b) with continuous stirring or sonication;

d) loading a carrier into a fluidized bed coater;

e) coating the carrier by spraying the solution of (c); and

f) drying the coated carrier particles to obtain the desired product.

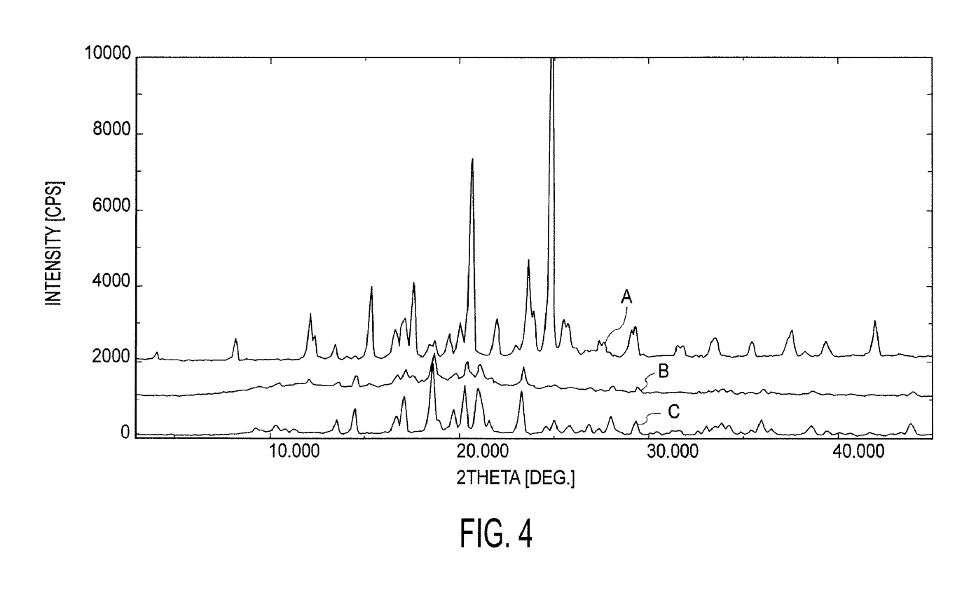

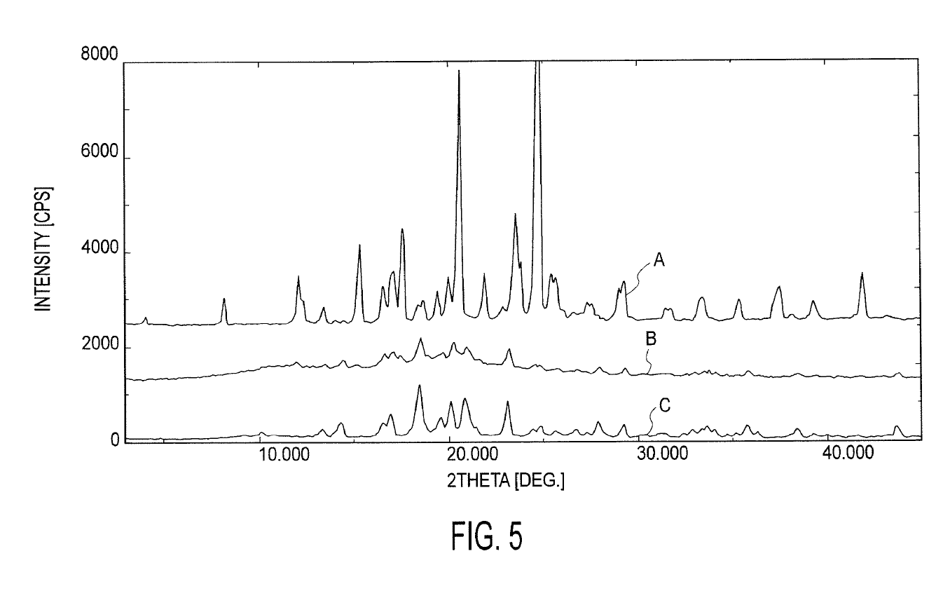

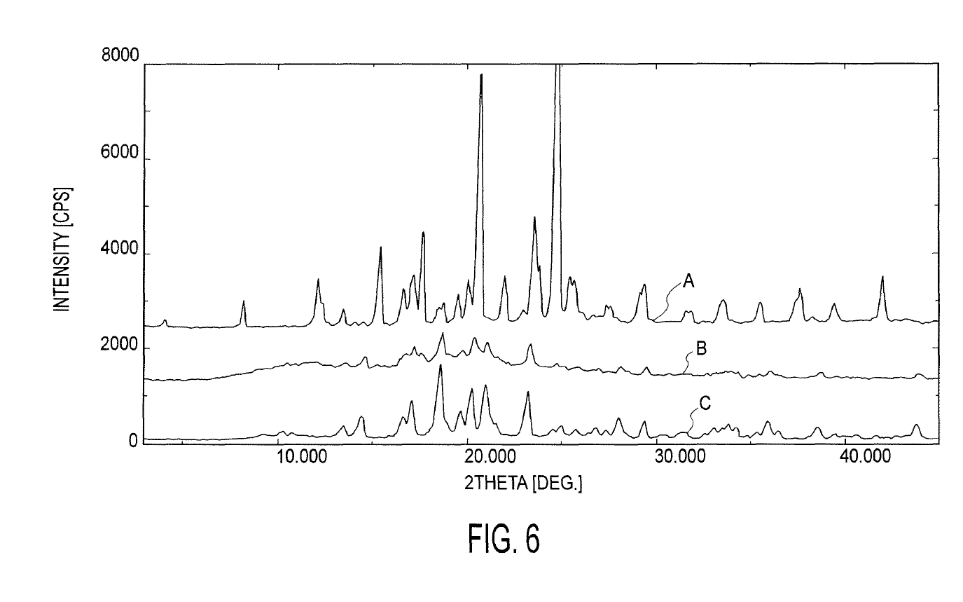

In an aspect, fluid bed coated stable and solubility-enhanced forms of aprepitant are subjected to storage stability testing at 25° C. and 60% RH, and 40° C. and 75% RH, for a commercially relevant time. The samples are examined by X-ray diffraction and compared with crystalline Form I and a placebo formulation. No peaks pertaining to crystalline Form I are detected, indicating absence of crystallinity in the pharmaceutical composition comprising the amorphous aprepitant co-precipitate (see FIGS. 4-6). Such solid state stability ensures the maintenance of enhanced solubility throughout the commercial shelf life of the formulation.

Formation of the inclusion complexes in solution can be characterized by techniques such as, for example, ultraviolet light spectroscopy, circular dichroism, fluorescence spectroscopy, nuclear magnetic resonance, potentiometry and the like. Solid inclusion complexes can be characterized by analytical techniques such as, for example, solubility in water or bio-relevant media, X-ray powder diffraction, differential scanning calorimetry, thermogravimetry, and the like.

In an aspect of the present invention, complexation with increasing concentrations of HPβCD significantly improves solubility characteristics of aprepitant. In yet another embodiment, complexation with varying concentrations of HPβCD wherein the pH is modified, further improves the aqueous solubility of aprepitant as compared to neutral pH conditions.

In an aspect of the present invention, the solubility-enhanced forms of aprepitant are in the form of fine particles of aprepitant. Fine particles include microparticle and nanoparticle preparations of aprepitant.

The term “nanosuspension” as used herein refers to suspensions comprising aprepitant in the form of nanoparticles.

The ‘unmilled form’ of aprepitant as used herein refers to aprepitant crystalline Form I and amorphous co-precipitate, as recovered from a manufacturing process, wherein the particle size has not been reduced.

In an aspect of the invention, amorphous co-precipitates of aprepitant are used for preparing micro- and nano-particles of aprepitant.

In another aspect of the invention, crystalline Form I of aprepitant is used to prepare microparticles of aprepitant that have effective average particle sizes from more than 3 μm to about 500 μm, or from about 10 μm to about 200 μm.

In an aspect, the invention provides processes for preparing microparticles of aprepitant, wherein an embodiment of a process comprises use of a Frizsch planetary ball mill loaded with zirconium beads. For a specific embodiment of preparation of microparticles, the ball mill can be rotated for about 30 to about 60 minutes at speeds of about 200 to about 600 rpm.

In another embodiment of the present invention, nanoaparticles of aprepitant amorphous co-precipitate are prepared by mixing with aqueous solutions of sodium lauryl sulphate, and nanoparticles of aprepitant crystalline Form I are prepared by mixing with aqueous solutions of sodium lauryl sulphate and adding a hydroxypropyl cellulose (e.g., Klucel™ LF). The dispersions are further subjected to particle size reduction such as by milling to obtain the desired particle size distribution.

In one embodiment of the present invention, a nanosuspension of aprepitant is dried to get a powdered composition.

Any method of solvent evaporation or drying is acceptable as long as it is not detrimental to the chemical stability of the drug as well as the solubilizing composition. Such methods include, for example, tray drying, vacuum drying, spray drying, spray coating, lyophilization, microwave drying and the like without limitation. Two or more methods can be used sequentially to ensure completeness of removal of the solvent medium or to achieve desired bulk properties of the dried solubilizing compositions.

In an embodiment, nanosuspensions of aprepitant as prepared above can be subjected to spray drying and the resulting powder is optionally further subjected to vacuum drying to reduce its moisture content.

According to an embodiment of the invention, a nanosuspension as prepared above can be further subjected to lyophilization to obtain a dry product, which constitutes one of the powder compositions of the invention. Lyophilization is a drying technique of particular interest in the preparation of dry powder compositions of the invention due to its rapid drying cycles, high throughputs, scalability and short exposure times to high temperatures, achievement of desired bulk properties, and other reasons.

In an embodiment, the invention includes characteristic properties of micro- and nanoparticles of aprepitant including particle size distribution, solubility, span, bulk density, tapped density, Hausner ratio, moisture content, aspect ratio, Carr index, and other parameters useful in the preparation of pharmaceutical compositions.

Formation of the micro- and nano-particles in solution can be characterized by techniques as for example scanning electron microscopy, transmission electron microscopy, nuclear magnetic resonance, polarized light microscopy, differential scanning calorimetry, X-ray diffraction, potentiometry and the like.

According to embodiments, a particle size distribution has particle sizes of substantially all of the aprepitant microparticles less than about 100 μm. A mean particle size ranges from 3 μm to about 50 μm, or from 3 μm to about 25 μm, or from about 3 μm to about 10 μm. In an embodiment, a mean particle size of aprepitant nanoparticles is less than about 2 μm, or less than about 0.5 μm.

In one embodiment of the present invention, D₉₀ of an amorphous aprepitant co-precipitate, as initially prepared, is about 7 times greater than that of crystalline Form I, as initially prepared. However, after micronization and nanonization, the particle sizes of amorphous co-precipitate are reduced to smaller sizes, as compared with those of crystalline Form I of aprepitant.

The term “particles” as used herein refers to individual particles of aprepitant.

Certain fine particle preparations of aprepitant show about 5-fold to about 200-fold, or from about 20-fold to about 150-fold, solubility enhancement when compared with uncomplexed aprepitant.

In an embodiment of the present invention the solubility of micro- and nano-particles of crystalline Form I of aprepitant is compared with that of aprepitant co-precipitates

In an aspect, amorphous co-precipitate, nanoparticles of amorphous co-precipitate and nanoparticles of crystalline Form I show comparable solubility, whereas nanoparticles of crystalline Form I of aprepitant show solubility enhancement about double, as compared with unmilled crystalline Form I, in 2.2% aqueous sodium lauryl sulphate solution. All of the tested samples show comparable solubility in the other test media.

In an embodiment, fine particles of aprepitant prepared by the above-described processes show about 0.5-fold to about 10-fold solubility enhancement, when compared with the unmilled aprepitant.

In a further embodiment, the enhancement of solubility of aprepitant in the compositions of the present invention results in significantly improved pharmacokinetic properties upon in vivo administration, in terms of faster absorption and more complete absorption defined by the bioavailability. The improved bioavailability may help to reduce the dose of aprepitant and thereby reduction in adverse effects may be achieved. Another aspect also provides aqueous solution formulations capable of intravenous administration.

In one embodiment, the pharmaceutical compositions of the present invention comprising solubility-enhanced forms of aprepitant, prepared by complexation with cyclodextrin, exhibit high stability against solid state conversions of aprepitant as compared with uncomplexed aprepitant.

In another embodiment, pharmaceutical compositions of the present invention comprise solubility-enhanced forms of aprepitant in the form of micro- and nano-particle preparations of aprepitant.

In an embodiment, pharmaceutical formulations of the present invention are solid dosage forms such as tablets, capsules, granules, pellets, beads, particles, mini-tablets, or orally disintegrating tablets, as well as liquid dosage forms like solutions, suspensions, syrups, and the like. The pharmaceutical formulations of the invention may be prepared using any process operations known in the art such as wet granulation, dry granulation, direct compression, spheronization, etc.

In a further embodiment, the pharmaceutical compositions of the present invention comprise at least one disintegrating agent.

In another embodiment, the pharmaceutical compositions of the present invention comprise at least two disintegrating agents, of which at least one is an ion exchange resin. Disintegrants include but are not limited to anionic resins such as DUOLITE™ AP143/1083 (cholestyramine resin USP), and cationic resins such as AMBERLITE™ IRP-64 (a porous copolymer of methacrylic acid crosslinked with divinylbenzene). In an embodiment, the pharmaceutical compositions of the present invention comprise at least two disintegrating agents, of which at least one is a cationic resin.

Other disintegrants include: natural starches such as maize starch and potato starch; directly compressible starches such as starch 1500; modified starches such as carboxymethyl starch and sodium starch glycolate; starch derivatives such as amylase; various grades of crospovidones; croscarmellose sodium; alginic acid and sodium alginate; microcrystalline celluloses; crosslinked polymers; crosslinked starches; and the like.

In an embodiment, pharmaceutical compositions of the present invention include excipients such as one or more of surfactants, emulsifiers. pH modulators, fillers, binders, diluents, glidants, lubricants, plasticizers, flavors, colorants, and the like. In an embodiment of the present invention, the pharmaceutical formulation of the present invention are in the form of orally disintegrating tablets that additionally comprise flavors, colors, film coating agents, and the like.

Surfactants improve the wettability of the active agent. Various useful surfactants include but are not limited to sodium lauryl sulfate, cetrimide, polysorbates such as polysorbate 80, poloxamers such as poloxamer 188 and poloxamer 407, sodium carboxy methylcelluloses, hydrogenated oils, polyoxyethylene glycols, polyoxypropylene glycols, polyoxyethylene sorbitan fatty acid esters, polyglycolized glycerides available commercially such as GELUCIRE® 40/14, GELUCIRE® 42/12, and GELUCIRE® 50/13, vitamin E TGPS, TWEEN® surfactants, SPAN® surfactants, and mixtures thereof.

Emulsifying agents can include any of a wide variety of cationic, anionic, zwitterionic, and amphoteric surfactants known in the art. Nonlimiting examples of anionic emulsifying agents include the alkoyl isothionates, alkyl and alkyl ether sulfates and salts thereof, alkyl and alkyl ether phosphates and salts thereof, alkyl methyl taurates, and alkali metal salts including sodium or potassium salts of long chain fatty acids.

Examples of amphoteric and zwitterionic emulsifying agents include but are not limited to carboxy, sulfonate, sulfate, phosphate, or phosphonate compounds. Examples are alkylimino acetates and iminodialkanoates and aminoalkanoates, imidazolinium and ammonium derivatives betaines, sultaines, hydroxysultaines, alkyl sarcosinates and alkanoyl sarcosinates, and the like.

Examples of suitable emulsifying agents include disodium cocoampho diacetate, oxyethylenated glyceryl cocoate (7 EO), PEG-20 hexadecenyl succinate, PEG-15 stearyl ether, the ricinoleic monoethanolamide monosulfosuccinate salts, oxyethylenated hydrogenated ricinoleic triglyceride, poloxamers, non-solid fatty substances such as sesame oil, almond oil, apricot stone oil, sunflower oil, octoxyglyceryl palmitate (or 2-ethylhexyl glyceryl ether palmitate), octoxyglyceryl behenate (or 2-ethylhexyl glyceryl ether behenate), dioctyl adipate, tartrates of branched dialcohols, and the like. Other useful non-ionic emulsifying agents include alkylene oxide esters of fatty acids, alkylene oxide diesters of fatty acids, alkylene oxide ethers of fatty alcohols, alkylene oxide esters, and the like.

Various useful diluents include but are not limited to different varieties and grades of starches like pregelatinized starches and maize starch, sugars such as lactose and sucrose, cellulose derivatives such as microcrystalline celluloses, and the like. Other useful diluents include but are not limited to carmelloses, sugar alcohols such as mannitol, sorbitol and xylitol, calcium carbonate, magnesium carbonate, dibasic calcium phosphate, and tribasic calcium phosphate.

Various useful binders include but are not limited to hydroxypropyl celluloses, hydroxypropyl methylcelluloses, polyvinylpyrrolidones, copovidones, powdered acacia, gelatin, guar gum, carbomers (e.g. Carbopol™), methylcelluloses, polymethacrylates, and starches.

Various useful glidants or anti-adherents include but are not limited to talc, silica derivatives, colloidal silicon dioxide and the like, and mixtures thereof.

Various plasticizers that can be used include but are not limited to castor oil, diacetylated monoglycerides, dibutyl sebacate, diethyl phthalate, glycerin, polyethylene glycols, propylene glycols, triacetin, and triethyl citrate.

Various lubricants that can be used include but are not limited to stearic acid and stearic acid derivatives such as magnesium stearate, calcium stearate, zinc stearate, sucrose esters of fatty acids, polyethylene glycols, talc, sodium stearyl fumarate, zinc stearate, castor oils, and waxes.

Other excipients particularly useful in making orally disintegrating dosage forms according to the present invention include sweetners, taste masking agents, flavors, colors, and the like.

Various useful coloring agents include but are not limited to iron oxides, which can be red, yellow, black or blends thereof.

In an embodiment, the present invention provides taste-masked compositions for oral administration as orally disintegrating or dissolving dosage forms. These formulations are useful for buccal or sublingual delivery of aprepitant. The taste-masked compositions of the present invention comprise one or more excipients selected from the group comprising resins, sweeteners, flavoring agents and the like.

In embodiments, an orally disintegrating pharmaceutical formulation containing aprepitant disintegrates in less than about 10 minutes upon immersion in water, when tested according to the method described hereinafter in Example 15.

In embodiments, orally disintegrating or dissolving compositons of the present invention comprise a solubility-enhanced form of aprepitant, a disintegrant and a resin.

In an embodiment, the orally disintegrating or dissolving compositions of the present invention further comprise sweeteners which include but are not limited to: natural sweeteners such as sucrose, dextrose, fructose, invert sugar, mannitol, sorbitol and the like; and synthetic sweeteners such as saccharin, aspartame, acesulfame potassium, cyclamates and the like. The amount of sweetener may vary depending on the sweetening strength of the particular sweetener used. Mixtures of any two or more sweeteners are useful in the invention.

Various useful flavoring agents include but are not limited to various fruit flavors, mint flavors and other natural or synthetic flavors.

An aspect of the present invention is further directed to processes for preparing pharmaceutical compositions containing aprepitant, wherein an embodiment of a process comprises:

a) Sifting drug, diluent, disintegrant and optionally other excipient(s) through a sieve.

b) Dry mixing sifted drug, diluent, disintegrant and other optional excipients.

c) Granulating the dry mix using a binder solution.

d) Drying the granules.

e) Passing the dried granules through a sieve.

f) Mixing the dried granules with sifted extragranular material(s).

The composition of step f) can be optionally compressed into tablets or can be filled into hard gelatin capsules.

Alternatively step b) may be blended with sifted extragranular materials and compressed into tablets or can be filled in hard gelatin capsules. Or step b) may be compacted and milled, then further blended with extragranular materials and compressed into tablets or filled into hard gelatin capsules.

In an embodiment, the invention includes physicochemical characteristics of the pharmaceutical compositions, wherein characteristics include particle size distribution, span, bulk density, Hausner ratio, moisture content, aspect ratio, Carr index, and the like that enhance effective delivery of aprepitant.

Pharmaceutical compositions of the present invention can be subjected to in vitro dissolution evaluations according to Test 711 “Dissolution” in United States Pharmacopoeia 29, United States Pharmacopeial Convention, Inc., Rockville, Md., 2005 (“USP”), to determine the release of drug from the dosage forms, and drug content can conveniently be determined in solutions by high performance liquid chromatography. The dissolution testing frequently is carried out using USP type II apparatus.

In further embodiment the in vitro dissolution studies are carried out using USP type II apparatus and 2.2% of sodium lauryl sulphate in purified water (“OGD media”), and/or fed state simulated intestinal fluid (FeSSIF) pH 5.0, as dissolution media with 75 rpm stirring. In an embodiment, pharmaceutical formulations of the present invention provide in vitro dissolution of aprepitant such that more than about 50% of the drug is dissolved within 60 minutes in 2.2% of sodium lauryl sulphate in purified water. In a further embodiment, pharmaceutical formulations of the present invention provide in vitro dissolution of aprepitant such that more than about 90% of the drug is dissolved within 60 minutes in 2.2% of sodium lauryl sulphate in purified water.

The formulation and preparation for the Fed State Simulated Intestinal Fluid (FeSSIF) biorelevant medium for dissolution testing comprises the following:

Sodium taurocholate, 15 mM.

Lecithin, 3.75 mM.

NaOH (pellets), 4.04 g.

Glacial acetic acid, 8.65 g.

NaCl, 11.874 g.

Purified water, q.s. to 1000 mL.

Media has a pH of 5.0 and an osmolality of about 670 mOsmol/kg.

Preparation of blank FeSSIF: Dissolve 20.2 g of NaOH (pellets), 43.25 g of glacial acetic acid, and 59.37 g of NaCl in purified water, and dilute to 5 L. Adjust the pH to exactly 5.0 using 1 N NaOH or 1 N HCl.

Preparation of FeSSIF: Dissolve 16.5 g of sodium taurocholate in 500 mL of blank FeSSIF. Add 59.08 mL of a solution containing 100 mg/mL lecithin in methylene chloride, forming an emulsion. The methylene chloride is eliminated under vacuum at about 40° C.: apply a vacuum for fifteen minutes at 250 mbar, followed by 15 minutes at 100 mbar. This results in a clear to slightly hazy, micellar solution having no perceptible odor of methylene chloride. After cooling to room temperature, adjust the volume to 2 L with blank FeSSIF. The recommended volume for simulating conditions in the upper small intestine after a meal is one liter.

In an embodiment, pharmaceutical formulations of the present invention provide in vitro dissolution of aprepitant such that more than about 30% of the drug is dissolved within 60 minutes in fed state simulated intestinal fluid pH 5.0 dissolution medium. In a further embodiment, pharmaceutical formulations of the present invention provide in vitro dissolution of aprepitant such that about 40% to about 80% of the drug is dissolved within 60 minutes in simulated intestinal fluid pH 5.0.

In an aspect, orally disintegrating or dissolving pharmaceutical formulations of aprepitant of the present invention release more than about 90% of the contained aprepitant within about 10 minutes, upon immersion in aqueous media having pH values about 4-8.

In one aspect of this embodiment, micronized aprepitant amorphous co-precipitate exhibits a comparable dissolution profile to that of a commercial aprepitant formulation (EMEND®).

In another aspect of the invention, micronized aprepitant crystalline Form I shows a comparable dissolution profile with that of an amorphous aprepitant co-precipitate, as prepared. However the dissolution profile can be much slower as compared with the micronized aprepitant amorphous co-precipitate and a commercial aprepitant formulation (EMEND®).

In an aspect, pharmaceutical formulations comprising a solid state stable non-nanoparticulate solubility-enhanced form of aprepitant according of the present invention provide a drug release profile, wherein about 15% to about 60% of the aprepitant is dissolved within about 15 minutes, about 25% to about 70% of the aprepitant is dissolved within about 30 minutes, about 35% to about 75% of the aprepitant is dissolved within about 45 minutes, and about 40% to about 90% of the aprepitant is dissolved within about 60 minutes, following immersion into 900 mL of Fed state simulated intestinal fluid pH 5.0 dissolution medium, when tested in USP apparatus II at 75 rpm stirring.

For an aspect of the invention, it can be concluded that, as aprepitant amorphous co-precipitate exhibits a comparable dissolution profile to a commercial aprepitant formulation, bioequivalent compositions may be prepared using micronized amorphous aprepitant co-precipitate.

A comparable dissolution profile in aqueous as well as bio-relevant media and in-vitro dissolution profile of size-reduced forms of aprepitant with a commercial aprepitant formulation suggests that using the size reduced forms of aprepitant can prepare bioequivalent pharmaceutical compositions. Such an enhancement in the aqueous and bio-relevant media solubility results in significantly improved pharmacokinetic properties and thereby bioavailability of arpepitant in the in vivo setting. The pharmaceutical compositions of the present invention may result in comparable plasma levels, tₘₐₓ, and area under the drug plasma concentration vs. time curve (AUC) of aprepitant to those of commercial formulations when administered orally.

In yet other aspects of the present invention, the lyophilized nanoparticle compositions of amorphous aprepitant co-precipitate as well as crystalline Form I of aprepitant exhibit higher rates of dissolution as compared with the commercial aprepitant formulation (EMEND®). The higher rate of dissolution in aqueous and bio-relevant media may significantly improve pharmacokinetic properties in the in vivo setting with faster absorption and faster onset of action, providing a reduced time after administration for attaining the maximum plasma aprepitant concentration (tₘₐₓ) and higher plasma levels of aprepitant when given orally, as well as more complete absorption defined by the bioavailability. Reduction in tₘₐₓ may reduce the time lapse to be maintained between administration of aprepitant and commencement of chemotherapy, or before inducing anesthesia for surgery. The improved bioavailability may help to reduce the dose of aprepitant and thereby a reduction in adverse effects may be achieved. Another aspect also provides aqueous solution formulations capable of intravenous administration.

In embodiments, pharmaceutical formulations of the present invention are appreciably stable and easy to manufacture using conventional processing steps, as compared to the currently marketed nanoparticulate formulations of aprepitant (EMEND®) that are prepared using difficult manufacturing steps and specialized machinery, but still provides in-vitro and in vivo release profiles of aprepitant that are comparable to those of EMEND® capsules.

In embodiments, in-vitro dissolution testing of the orally disintegrating pharmaceutical formulations of the present invention provides release of more than 90% of conatined aprepitant within 10 minutes.

The present invention also provides methods of use of solubility-enhanced forms of aprepitant, solid state stable forms of aprepitant, solid state stable solubility-enhanced forms of aprepitant, and pharmaceutical formulations thereof in the management (prophylaxis, amelioration and/or treatment) of chemotherapy induced nausea and vomiting, comprising administering to a subject in need thereof an effective amount of aprepitant.

In an embodiment the invention includes use of product packaging materials such as containers and lids of HDPE, low-density polyethylene (LDPE) and/or polypropylene and/or glass, and blisters or strips composed of aluminum, high-density polypropylene, polyvinyl chloride, and/or polyvinylidene dichloride. The described packaging materials are only representative, as many other materials will be suitable.

Certain specific aspects and embodiments of the invention will be further described in the following examples, which are provided solely for purposes of illustration and are not intended to limit the scope of the invention in any manner.

### Example 1

### Solubility of Aprepitant and its Complexes

Crystalline Form I of aprepitant, amorphous aprepitant, or aprepitant coprecipitate was added to water and to aqueous HPβCD solutions of different concentrations, with stirring until the saturation solubility was reached. Table 1 shows the solubility obtained, where the pH conditions are:

1A—pH 7.0.

1B—pH adjusted to 1.2 using hydrochloric acid.

1C—pH adjusted to 11 with 0.3% sodium bicarbonate solution.

TABLE 1

| | Solubility (mg/mL) | | |
|---|---|---|---|
| Composition | 1A | 1B | 1C |
| Crystalline aprepitant Form I | 0.0005 | 0.0005 | 0.0005 |
| Amorphous aprepitant coprecipitate | 0.001 | 0.001 | 0.001 |
| Amorphous aprepitant | 0.008 | 0.191 | 0.61 |
| coprecipitate-2.5% HPβCD complex | | | |
| Crystalline aprepitant Form I-2.5% | 0.004 | 0.1 | 0.375 |
| HPβCD complex | | | |
| Amorphous aprepitant | 0.017 | 0.307 | 1.275 |
| coprecipitate-5% HPβCD complex | | | |
| Crystalline aprepitant Form I-5% | 0.008 | 0.168 | 0.782 |
| HPβCD complex | | | |
| Amorphous aprepitant | 0.031 | 0.617 | 1.483 |
| coprecipitate-10% HPβCD complex | | | |
| Crystalline aprepitant Form I-10% | 0.019 | 0.318 | 0.958 |
| HPβCD complex | | | |
| Amorphous aprepitant | 0.054 | 0.895 | 2.097 |
| coprecipitate-15% HPβCD complex | | | |
| Crystalline aprepitant Form I-15% | 0.026 | 0.455 | 1.265 |
| HPβCD complex | | | |
| Amorphous aprepitant | 0.081 | 1.255 | 2.45 |
| coprecipitate-20% HPβCD complex | | | |
| Crystalline aprepiatnt Form I-20% | 0.043 | 0.611 | 1.483 |
| HPβCD complex | | | |
| Amorphous aprepitant | 0.106 | 1.662 | 2.811 |
| coprecipitate-25% HPβCD complex | | | |
| Crystalline aprepitant Form I-25% | 0.058 | 0.757 | 1.698 |
| HPβCD complex | | | |
| Amorphous aprepitant | 0.163 | 2.319 | 2.987 |
| coprecipitate-30% HPβCD complex | | | |
| Crystalline aprepitant Form I-30% | 0.066 | 0.878 | 0.859 |
| HPβCD complex | | | |

The amorphous aprepitant co-precipitate is prepared using the following method:

1 g of aprepitant and 1 g of povidone (PVP K30) are dissolved in 200 mL of dichloromethane with heating to 40° C. The solution is filtered in the hot condition and the dichloromethane is removed using distillation in a Buchi Rotavapor apparatus under a vacuum of 0-20 torr. 1.8 g of a dried coprecipitate of aprepitant with povidone is obtained.

Complexes with HPβCD are prepared by adding aprepitant to aqueous solutions of HPβCD and sonicating the solutions to dissolve aprepitant, until saturation is attained.

The following examples represent pharmaceutical compositions of the present invention.

### Example 2

### Composition of Aprepitant with HPβCD and Sodium Lauryl Sulphate

| Ingredient | mg/mL |
|---|---|
| Aprepitant | 40 |
| HPβCD | 80 |
| Sodium lauryl sulphate | 20 |

Manufacturing Process:

1. Mix aprepitant, HPβCD and sodium lauryl sulfate together and sift through an ASTM #40 mesh sieve.

2. Place the above sifted physical mixture into a vessel, add water, and sonicate for 1 hour to obtain a clear solution.

3. Filter the solution through a 0.22 μm membrane filter.

### Example 3

### Dry Powder Composition for Aprepitant 40 mg Capsules

| Ingredient | mg/Capsule |
|---|---|
| Aprepitant | 40 |
| HPβCD | 80 |
| Mannitol | 10 |
| Sodium carbonate | 1 |
| Acetonitrile* | 30 mL |
| Water* | 10 mL |

*Evaporates during processing.

Manufacturing Process:

1) Dissolve aprepitant in acetonitrile at 75° C.

2) Add HPβCD, mannitol and sodium carbonate to water and stir until dissolved.

3) Add drug solution from step 1 to cyclodextrin solution of step 2, with continuous stirring at about 50° C. to allow complexation.

4) Remove acetonitrile by distillation under vacuum.

5) Filter the residue solution through a 0.22 μm membrane filter.

6) Dry the residue solution by spraying it onto pharmacologically inert particulate material and further subject to vacuum drying to reduce moisture

7) Fill the dried product into hard gelatin capsules.

### Example 4

### Pharmaceutical Formulation Using Spray Drying

| | Weight Percent | |
|---|---|---|
| | 4A (Drug | 4B (Placebo |
| Ingredient | Formulation) | Formulation) |
| Aprepitant amorphous co-precipitate*# | 32.26 | — |
| PVP-K30 | — | 19.23 |
| Hydroxypropyl-β-cyclodextrin# | 16.13 | 19.23 |
| Mannitol (Pearlitol ™ SD200)# | 32.26 | 38.46 |
| Croscarmellose sodium | 10.97 | 13.07 |
| Microcrystalline cellulose | 8.39 | 10 |
| (Avicel ™ PH112) | | |
| Water‡ | q.s. | q.s. |
| Acetonitrile‡ | q.s. | q.s. |

*Aprepitant amorphous co-precipitate as prepared in Example 1.
#These components are used as spray dried forms.
‡Evaporates during processing.

Manufacturing Process:

1. Aprepitant amorphous co-precipitate is dissolved in acetonitrile with sonication.

2. Hydroxypropyl-β-cyclodextrin is dissolved in water.

3. Both the solutions are mixed with sonication. Ratio of acetonitrile to water is 1:1.

4. Mannitol is dissolved in the solution from step 3 and sonicated for 10 minutes.

5. The solution is spray dried.

6. The spray dried complex of step 5 is blended with croscarmellose sodium and Avicel PH112.

7. The composition of step 6 is filled into hard gelatin capsules, such that each capsule contains 125 mg of aprepitant. Alternatively the composition of step 6 is compressed to form a tablet.

For Example 4B, manufacturing process is similar to 4A except that the acetonitrile did not contain any co-precipitate.

In vitro dissolution testing of a formulation of Example 4A is performed using the USP procedure and the following parameters, and is compared with EMEND®. The data are shown in Table 2.

Apparatus: USP Type II (paddle).

Paddle speed: 75 rpm.

Medium: 900 mL of 2.2% SLS in purified water.

TABLE 2

| | |
|---|---|
| | Cumulative % of |
| Time | Drug Dissolved |

| (minutes) | EMEND 125 mg | Example 4A |
|---|---|---|
| 15 | 57 | 36 |
| 20 | 62 | 49 |
| 30 | 69 | 75 |
| 45 | 76 | 99 |
| 60 | 81 | 103 |

### Example 5

### Pharmaceutical Formulation of Stable and Solubility-Enhanced Aprepitant with Fluidized Bed Coating

| | Weight Percent | | | | | |
|---|---|---|---|---|---|---|
| | Drug-Containing | | | | | |
| | Formulations | | | Placebo Formulations | | |
| Ingredient | 5A | 5B | 5C | 5D | 5E | 5F |
| Aprepitant amorphous | 35.71 | 36.76 | 36.76 | — | — | — |
| co-precipitate*# | | | | | | |
| Povidone K30 | — | — | — | 21.74 | 22.52 | 22.52 |
| Hydroxypropyl-β- | 17.86 | 18.38 | 18.38 | 21.74 | 22.52 | 22.52 |
| cyclodextrin# | | | | | | |
| Mannitol (Pearlitol | 17.86 | 18.38 | 18.38 | 21.74 | 22.52 | 22.52 |
| SD200)# | | | | | | |
| Microcrystalline cellulose | 4 | 4.12 | 4.12 | 4.87 | 5.05 | 5.05 |
| (Avicel PH 112) | | | | | | |
| Sodium starch glycolate | 14.29 | 14.71 | 5.15 | 17.39 | 18.02 | 6.31 |
| Crospovidone XL 10 | 9.29 | — | 9.56 | 11.3 | — | 11.71 |
| Amberlite ™ IRP88† | — | 6.62 | 6.62 | — | 8.11 | 8.11 |
| Magnesium stearate | 0.71 | 0.74 | 0.74 | 0.87 | 0.9 | 0.9 |
| Water‡ | q.s. | q.s. | q.s. | q.s. | q.s. | q.s. |
| Acetonitrile‡ | q.s. | q.s. | q.s. | q.s. | q.s. | q.s. |

*Aprepitant amorphous co-precipitate as prepared in Example 1.
#These components are used as spray dried forms.
†Amberlite IRP88 is a cationic ion exchange resin.
‡Evaporates during processing.

Manufacturing Process:

1. Aprepitant amorphous co-precipitate is dissolved in acetonitrile with sonication.

2. Hydroxypropyl-β-cyclodextrin is dissolved in water.

3. Both the solutions are mixed with sonication. Ratio of acetonitrile to water is 1:1, by volume.

4. Mannitol particles are loaded into a fluidized bed coater (FBC).

5. The contents of step 3 are coated onto mannitol.

6. After spraying, the contents of FBC are dried and the complex is passed through an ASTM #30 mesh sieve.

7. The complex of step 6 is blended together with Avicel PH 112, sodium starch glycolate, crospovidone, Amberlite and magnesium stearate.

8. The composition of step 7 is filled into hard gelatin capsules, such that each capsule contains 125 mg of aprepitant. Alternatively the composition of step 7 is compressed to form tablets.

For Example 5D, 5E and 5F, manufacturing process is similar to 5A, 5B and 5C respectively except that the acetonitrile did not contain any co-precipitate.

In vitro dissolution testing of a formulation of Example 5B as described herein is performed using the USP procedure and the following parameters and is compared with EMEND®. The data are shown in Table 3.

Apparatus: USP Type II (paddle).

Paddle speed: 75 rpm.

Medium: 900 mL of Fed state simulated intestinal fluid (FeSSIF), pH 5.0.

TABLE 3

| | |
|---|---|
| | Cumulative % of |
| Time | Drug Dissolved |

| (minutes) | EMEND ® 125 mg | Example 5B |
|---|---|---|
| 15 | 52 | 18 |
| 20 | 54 | 27 |
| 30 | 58 | 38 |
| 45 | 59 | 50 |
| 60 | 60 | 59 |

### Example 6

### Pharmaceutical Formulation of Stable and Solubility-Enhanced Aprepitant Using Fluidized Bed Coating

Weight
Percent

| Ingredient | 6A | 6B |
|---|---|---|
| Aprepitant amorphous co-precipitate* | 34.34 | 35.31 |
| Hydroxypropyl-β-cyclodextrin | 17.17 | 17.65 |
| Mannitol (Pearlitol SD200) | 17.17 | 17.65 |
| Microcrystalline cellulose (Avicel PH112) | 3.85 | 3.95 |
| Sodium starch glycolate | 13.74 | 11.3 |
| Crospovidone XL10 | 8.93 | 9.18 |
| Sodium carbonate | 4.12 | 4.24 |
| Magnesium stearate | 0.68 | 0.71 |
| Water‡ | q.s. | q.s. |
| Acetonitrile‡ | q.s. | q.s. |

*Aprepitant amorphous co-precipitate as prepared in Example 1.
‡Evaporates during processing.

Manufacturing process: similar to that described in Example 5.

In vitro dissolution testing of a formulation of Example 6A as described herein is performed using the following parameters and is compared with EMEND®. The data are shown in Table 4.

Apparatus: USP Type II (paddle).

Paddle speed: 75 rpm.

Volume: 900 mL.

TABLE 4

| | Cumulative % of Drug Dissolved | | | |
|---|---|---|---|---|
| | 2.2% Sodium | | Fed State | |
| | Lauryl Sulphate | | Simulated Intestinal | |
| | in Purified Water | | Fluid (FeSSIF) pH 5.0 | |
| Time | EMEND ® | | EMEND ® | |
| (minutes) | 125 mg | Example 6A | 125 mg | Example 6A |
| 15 | 57 | 57 | 52 | 34 |
| 20 | 62 | 78 | 54 | 45 |
| 30 | 69 | 98 | 58 | 56 |
| 45 | 76 | 101 | 59 | 63 |
| 60 | 81 | 102 | 60 | 67 |

### Example 7A

### Microparticles of Aprepitant

1) Aprepitant amorphous co-precipitate as prepared in Example 1 is loaded into a Fritzsch planetary ball mill, previously loaded with zirconium beads.

2) The mill is loaded with 12 balls.

3) The ball mill is rotated at about 300 rpm for about 30 minutes to prepare microparticles of aprepitant co-precipitate.

### Example 7B

### Microparticle Composition of Aprepitant Amorphous Co-Precipitate

1) Microparticles of Example 7A, lactose monohydrate and microcrystalline cellulose PH101 are sifted through an ASTM #20 mesh sieve.

2) The sifted ingredients of step 1) are blended together in a rapid mixer granulator for about 10 minutes to form the microparticle composition.

### Example 8

### Composition of Aprepitant Crystalline Form I

A composition of aprepitant crystalline Form I was prepared by a process similar to that described in Examples 7A and 7B for a microparticle composition of aprepitant amorphous co-precipitate

### Example 9A

### Nanosuspension of Aprepitant Amorphous Co-Precipitate

1) 0.325 g of sodium lauryl sulphate is dissolved in 500 mL of water.

2) 25 g of aprepitant amorphous co-precipitate as prepared in Example 1 is slowly added to the solution of step 1) and stirred using an overhead mechanical stirrer for about 30 minutes.

3) The suspension formed in step 2) is circulated through a bead mill containing yttrium-stabilized zirconium beads (0.2-0.3 mm diameter) and milled for about 90 minutes at 6° C. to obtain the desired particle size distribution.

### Example 9B

### Nanosuspension of Aprepitant Crystalline Form I

A nanosuspension of aprepitant crystalline Form I is prepared using a process similar to that described in Example 9A for an amorphous co-precipitate of aprepitant. The process includes addition of 2.5 g of hydroxypropyl cellulose (Klucel LF) to the solution of step 1) before addition of aprepitant crystalline Form I.

### Example 10A

### Lyophilization of a Nanosuspension of Aprepitant Amorphous Co-Precipitate

1) 148.5 g of aprepitant nanosuspension of Example 9A is placed in a glass beaker and 1.5 g of lactose monohydrate is added and stirred well.

2) The suspension of step 1) is subjected to lyophilization for about 48 hours at a temperature about −30° C.

3) 1 g of the lyophilized powder is mixed with 0.5 g of microcrystalline cellulose PH101.

### Example 10B

### Lyophilization of a Nanosuspension of Aprepitant Crystalline Form I

Lyophilization of a nanosuspension of aprepitant crystalline Form I is carried out by a process similar to that described in Example 10A for an amorphous co-precipitate of aprepitant.

### Example 11

### Particle Size Analysis of Aprepitant Particles

Particle size analysis was carried out using a Malvern Mastersizer. The particle size distributions are shown in Table 5.

TABLE 5

| | Particle Size (μm) | | | | |
|---|---|---|---|---|---|
| | Crystalline Form I | | Amorphous Co-precipitate | | |
| | | Example | Example | Example | Example |
| Parameter | Unmilled | 10B | 1 | 7B | 10A |
| D₉₀ | 52.0 | 0.392 | 388.5 | 55.0 | 0.194 |
| D₅₀ | 29.2 | 0.145 | 197.1 | 7.25 | 0.12 |
| D₁₀ | 17.1 | 0.069 | 59 | 0.25 | 0.077 |

### Example 12

### Dissolution Profiles of Microparticle Aprepitant Compositions

The dissolution profiles of quantities providing 125 mg of aprepitant from a microparticle composition of Example 7B, amorphous co-precipitate of Example 1, and crystalline Form I of Example 8 are compared with a commercial formulation of aprepitant. Table 6 shows the comparative dissolution profiles.

TABLE 6

| Time | | | | |
|---|---|---|---|---|
| (minutes) | EMEND* | Example 8 | Example 1 | Example 7B |
| 5 | 83 | 41 | 36 | — |
| 10 | 87.5 | 55 | 45.5 | 99 |
| 15 | 90 | 63 | 54 | 100 |
| 20 | 91.5 | 69 | 60 | 100 |
| 30 | 93.5 | 75.5 | 69 | 100 |
| 45 | 95.5 | 81 | 78 | 100 |
| 60 | 97 | 84 | 85 | 100 |

*Contents of an EMEND 125 mg capsule.

Method from USP, with the conditions:

Apparatus: USP type II (paddle).

Medium: 900 mL of 2.2% sodium lauryl sulphate in purified water.

Speed: 100 rpm.

The micronized aprepitant amorphous co-precipitate gives comparable in-vitro dissolution profile to the commercial formulation of aprepitant, whereas crystalline Form I as well as that of amorphous co-precipitate show a slower rate of dissolution.

### Example 13

### Dissolution Profiles of Lyophilized Aprepitant Compositions

The dissolution profiles of lyophilized compositions of Example 10 (in amounts providing 125 mg of aprepitant) are compared with a commercial formulation of aprepitant. Table 7 shows the comparative dissolution profiles.

Method from USP, with the conditions:

Apparatus: USP type II (paddle method).

Volume: 900 mL.

TABLE 7

| | 0.5% Sodium Lauryl Sulphate | | | Fed State Simulated Intestinal | | |
|---|---|---|---|---|---|---|
| | in Purified Water (50 rpm) | | | Fluid (FeSSIF) pH 5.0 (100 rpm) | | |
| Time | | Example | Example | | Example | Example |
| (minutes) | EMEND* | 10B | 10A | EMEND* | 10B | 10A |
| 5 | 63 | 100 | 102 | 63.5 | 70 | 60.5 |
| 10 | 70 | 100 | 100 | 64 | 71 | 62 |
| 15 | 75 | 100 | 101 | 65 | 72 | 62.5 |
| 20 | 78 | 100 | 101 | 65 | 72 | 62.5 |
| 30 | 84 | 100 | 101 | 65.5 | 73 | 63 |
| 45 | 89 | 100 | 101 | 63.5 | 73 | 63.5 |
| 60 | 92 | 100 | 101 | 63.5 | 70 | 60.5 |

*Contents of an EMEND 125 mg capsule.

The compositions of aprepitant amorphous co-precipitate and crystalline Form I have higher rates of dissolution as compared with the commercial formulation of aprepitant.

The nanoparticle compositions of aprepitant amorphous co-precipitate as well as that of crystalline Form I have higher rates of dissolution as compared with the commercial formulation of aprepitant in fed state simulated intestinal fluid.

### Example 14

### Aprepitant-HPβCD Complex Using Aprepitant Form 1

| Ingredient | Weight Percent |
|---|---|
| Aprepitant crystalline (Form I) | 33.33 |
| Mannitol (Pearlitol SD 200) | 33.33 |
| HPβCD (Kleptose HPB) | 33.33 |
| Acetonitrile* | q.s. |
| Water* | q.s. |

*Evaporates during processing.

Manufacturing Process:

1. Crystalline aprepitant (Form I) is dissolved with sonication in acetonitrile and hydroxypropyl-β-cyclodextrin is dissolved in water. The solutions are mixed with sonication for 10 minutes to produce a clear solution. Acetonitrile and water are in a 2:1 proportion, by volume.

2. Mannitol (Pearlitol SD 200) is loaded into a FBP (fluidized bed processor).

3. Sonicated solution of step 1 is sprayed onto mannitol in the FBP using top spray. FBP parameters:

Inlet temperature: 70-75° C.

Product temperature: 35-40° C.

Exhaust temperature: 29° C.

Blower drive speed: 20-22.

Spray pump speed (rpm): 8-10.

Atomization air (bar): 0.8-1.0.

Air flow (cfm): 199.

4. After spraying, the samples are dried in the FBP for 10 minutes.

### Example 15

### Orally Disintegrating Dosage form of Aprepitant-HPβCD Complex Using Crystalline Aprepitant Form 1

| | Weight Percent | | |
|---|---|---|---|
| Ingredient | 15A | 15B | 15C |
| Aprepitant-HPβ3CD complex (Example 14) | 60.68 | 53.52 | 52.81 |
| Avicel PH112 | 4.3 | 13.55 | 13.37 |
| Sodium starch glycolate (Primojel ™) | 15.36 | — | — |
| Crospovidone XL10 | 9.98 | 4.06 | 4.01 |
| Sodium lauryl sulphate | 6.14 | — | — |
| Amberlite IRP88 (polacrilin potassium) | — | 8.13 | 8.02 |
| Mannitol | — | 13.55 | 13.37 |
| Aerosil 200 (colloidal silicon dioxide) | — | — | 4.01 |
| Povidone (PVP K 30) | — | — | 1.38 |
| Magnesium stearate | 0.77 | 0.68 | 0.67 |
| Aspartame | 1.23 | 1.08 | 1.07 |
| Strawberry flavour | 1.54 | 1.36 | 1.38 |

Manufacturing Process:

1. All ingredients are passed through a #30 mesh sieve.

2. Aprepitant-HPβCD complex is blended together with the required Primojel, crospovidone, sodium lauryl sulphate, Amberlite, mannitol, Aerosil, and povidone ingredients, and mixed well.

3. Avicel PH112 is added to the blends of step 2 and mixed well.

4. Aspartame and strawberry flavour are added to the blends of step 3 and mixed well.

5. The blend of step 4 is combined with magnesium stearate.

6. The lubricated blend of step 5 is compressed to form tablets.

The disintegration times of orally disintegrating tablets is determined using the general procedure of Test 701 “Disintegration” in United States Pharmacopeia 29, with a disintegration test apparatus (Electrolab ED2L, Mumbai, India). The apparatus has a basket-rack assembly supporting 6 cylindrical glass tubes with 21.5-mm internal diameter, oscillated vertically over 55 mm in distilled water in a beaker of 1000 mL capacity at 37±2° C. at 30 cycles/minute. The openings of the mesh at the bottom of the glass tubes are 2 mm. Tablets are considered disintegrated when completely dispersed fragments are obtained. Table 8 shows disintegration times of the orally disintegrating tablets of Example 15.

TABLE 8

| Example | Disintegration Time (seconds) |
|---|---|
| 15A | 190 |
| 15B | 50 |
| 15C | 105 |

1. A solid state non-nanoparticulate solubility-enhanced form of aprepitant comprising aprepitant as a co-precipitate, premix, or a solid dispersion with at least one pharmaceutically acceptable carrier, wherein the said co-precipitate, premix, or a solid dispersion of aprepitant is prepared in the form of an inclusion complex with at least one cyclodextrin or cyclodextrin derivative.

2. The solid state non-nanoparticulate solubility-enhanced form of aprepitant of claim 1, wherein aprepitant is present as an amorphous co-precipitate.

3. The solid state non-nanoparticulate solubility-enhanced form of aprepitant of claim 2, wherein the amorphous co-precipitate comprises aprepitant and polyvinylpyrrolidone as a carrier, in a weight ratio from about 4:1 to about 1:4.

4. The solid state non-nanoparticulate solubility-enhanced form of aprepitant of claim 2, wherein the amorphous co-precipitate comprises aprepitant and polyvinylpyrrolidone as a carrier in a weight ratio of about 1:1.

5. The solid state non-nanoparticulate solubility-enhanced form of aprepitant of claim 2, wherein an amorphous co-precipitate is prepared by a process comprising removing solvent from a solution comprising aprepitant and a pharmaceutically acceptable carrier.

6. The solid state non-nanoparticulate solubility-enhanced form of aprepitant of claim 1 that provides from about 2to about 100-fold higher solubility in aqueous media, as compared with crystalline aprepitant.

7. The solid state non-nanoparticulate solubility-enhanced form of aprepitant of claim 1, wherein a weight ratio of aprepitant to cyclodextrin ranges from about 1:0.01 to about 1:200.

8. The solid state non-nanoparticulate solubility-enhanced form of aprepitant of claim 1, wherein a weight ratio of aprepitant to cyclodextrin ranges from about 1:0.1 to about 1:100.

9. The solid state non-nanoparticulate solubility-enhanced form of aprepitant of claim 1, wherein a weight ratio of aprepitant to cyclodextrin ranges from about 1:0.25 to about 1:50.

10. A pharmaceutical formulation comprising a solid state non-nanoparticulate solubility-enhanced form of aprepitant of claim 1 and one or more pharmaceutically acceptable excipients.

11. A process for preparing an inclusion complex of aprepitant with a cyclodextrin, or a derivative of a cyclodextrin, comprising combining aprepitant or a co-precipitate, premix, or a solid dispersion of aprepitant with a cyclodextrin or cyclodextrin derivative in a solution having a pH within the ranges of about 1 to about 5, or about 8 to about 12.

12. The process of claim 11, wherein aprepitant or a co-precipitate, premix, or a solid dispersion of aprepitant is added to the solution in solid form.

13. The process of claim 11, wherein aprepitant or a co-precipitate, premix, or a solid dispersion of aprepitant is added to the solution in amorphous form.

14. The process of claim 11, wherein a weight ratio of aprepitant or a co-precipitate, premix, or a solid dispersion of aprepitant to cyclodextrin or cyclodextrin derivative is between about 1:0.01 and about 1:200.

15. The process of claim 11, wherein a weight ratio of aprepitant or a co-precipitate, premix, or a solid dispersion of aprepitant to cyclodextrin or cyclodextrin derivative is between about 1:0.1 and about 1:100.

16. The process of claim 11, wherein a weight ratio of aprepitant or a co-precipitate, premix, or a solid dispersion of aprepitant to cyclodextrin or cyclodextrin derivative is between about 1:0.25 and about 1:50.

17. The process of claim 11, wherein the solution comprises water and an organic solvent.

18. The process of claim 17, wherein the solution comprises water and at least one organic solvent in a volume ratio between about 1:50 and about 50:1.

19. The process of claim 17, wherein the solution comprises water and at least one organic solvent in a volume ratio between about 1:10 and about 10:1.

20. The process of claim 17, wherein the solution comprises water and at least one organic solvent in a volume ratio of about 1:1.

21. The process of claim 11, wherein the pH of the solution is about 1 to about 5.

22. The process of claim 11, wherein the pH of the solution is about 8 to about 12.

23. A pharmaceutical formulation comprising a solid state stable non-nanoparticulate solubility-enhanced form of aprepitant, wherein more than about 30% of the aprepitant is dissolved within 60 minutes after immersion in 900 ml of fed state simulated intestinal fluid pH 5.0 dissolution medium, when tested in USP apparatus II at 75 rpm stirring.

24. The pharmaceutical formulation of claim 23, wherein about 40% to about 80% of the aprepitant is dissolved within 60 minutes.

25. A pharmaceutical formulation comprising a solid state stable non-nanoparticulate solubility-enhanced form of aprepitant, wherein about 15% to about 60% of the aprepitant is dissolved within about 15 minutes, about 25% to about 70% of the aprepitant is dissolved within about 30 minutes, about 35% to about 75% of the aprepitant is dissolved within about 45 minutes, and about 40% to about 90% of the aprepitant is dissolved within about 60 minutes, after immersion into 900 ml of fed state simulated intestinal fluid pH 5.0 dissolution medium, when tested in USP apparatus II at 75 rpm stirring.

26. An orally disintegrating pharmaceutical formulation, containing a solubility-enhanced form of aprepitant, that disintegrates in less than about 10 minutes upon immersion in water.

27. The orally disintegrating or dissolving pharmaceutical formulation of claim 26, comprising aprepitant in the form of an inclusion complex with at least one cyclodextrin or derivative of cyclodextrin.

28. The orally disintegrating or dissolving pharmaceutical formulation of claim 26, comprising at least one disintegrant, and optionally a resin.

29. The orally disintegrating or dissolving pharmaceutical formulation of claim 26, containing at least one taste masking ingredient.

30. A lyophilized pharmaceutical composition comprising a solubility-enhanced form of aprepitant of claim 1.

31. A method of treatment or prevention of emesis, comprising orally administering to a patient in need thereof a pharmaceutical formulation comprising a solubility-enhanced form of aprepitant of claim 1, alone or in combination with a corticosteroid, a 5-HT receptor antagonist, or both.

32. The method of claim 31, wherein the emesis is associated with cancer chemotherapy or post-operative condition.
